(19) United States
(12) Patent Application Publication
(10) Pub. No.: US 2016/0168126 A1
(43) Pub. Date: Jun. 16, 2016

(54) CHEMICAL COMPOUNDS

(71) Applicants: SYNGENTA LIMITED, Guildford, Surrey (GB); SYNGENTA PARTICIPATIONS AG, Basel (CH)

(72) Inventors: Alan John Dowling, Bracknell, Berkshire (GB); Timothy Robert Desson, Bracknell, Berkshire (GB); William Guy Whittingham, Bracknell, Berkshire (GB); Anne Jacqueline Dalencon, Bracknell, Berkshire (GB); James Alan Morris, Bracknell, Berkshire (GB); Jutta Elisabeth Boehmer, Bracknell, Berkshire (GB); Mangala Phadte, Corlim, Ilhas (IN); Adrian Longstaff, Bracknell, Berkshire (GB); Matthew Brian Hotson, Bracknell, Berkshire (GB); Paul John De Fraine, Bracknell, Berkshire (GB); Regis Jean Georges Mondiere, Stein (CH); Shuji Hachisu, Bracknell, Berkshire (GB); Alison Jane Thompson, Bracknell, Berkshire (GB); Vijaya Gopal Gopalsamuthiram, Corlim, Ilhas (IN)

(73) Assignees: SYNGENTA LIMITED, Guildford, Surrey (GB); SYNGENTA PARTICIPATIONS AG, Basel (CH)

(21) Appl. No.: 14/907,342

(22) PCT Filed: Aug. 5, 2013
(86) PCT No.: PCT/EP2013/066395

§ 371 Date: Jan. 25, 2016

(51) Int. Cl. C07D 403/04; C07D 403/14; C07D 513/04; A01N 43/90; C07D 471/04; A01N 43/56; C07D 487/04

(52) U.S. Cl. CPC C07D 403/04; A01N 43/56; C07D 403/14; C07D 487/04; A01N 43/90; C07D 471/04; C07D 513/04

(57) ABSTRACT

The invention relates to pyrrolone compounds of the formula (I) wherein X, R^{a}, R^{b}, R^{c}, R¹, R² and R³ are as defined in the specification. Furthermore, the present invention relates to processes and intermediates for making compounds of formula (I), to herbicidal compositions comprising these compounds and to methods of using these compounds to control plant growth.

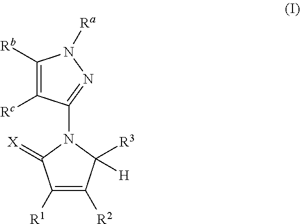

The present invention relates to certain substituted pyrrolone derivatives, to processes for their preparation, herbicidal compositions comprising them, and their use in controlling plants or inhibiting plant growth.

Herbicidal pyrrolones of the formula

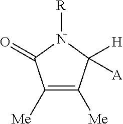

wherein A is hydroxy, halogen or OAcyl; and R is an optionally substituted aryl, aralkyl or heteroaryl group are taught in Swiss patent application CH633678.

Further herbicidal pyrrolones of the formula

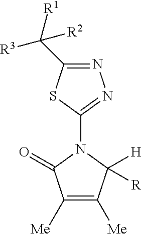

wherein R is inter alia OH, R¹ is H or alkyl, and R² and R³ are alkyl, haloalkyl, or alkylene are taught in EP0286816A1.

Further herbicidal pyrrolones of the formula

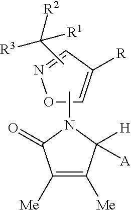

wherein A is e.g. OH, R is H, halogen, alkyl, haloalkyl, or alkoxyl, R¹ to R³ are each H, halogen, alkyl, haloalkyl, alkyoxyalkyl, or R² and R³ together form a 3 to 7 membered ring; are disclosed in EP0297378A2.

Further herbicidal pyrrolones of the formula

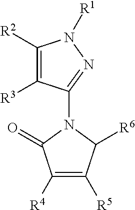

wherein R¹ is H, alkyl, haloalkyl, alkenyl, haloalkenyl, alkynyl, alkoxyalkyl or optionally substituted aryl or aralkyl, R² is H, alkyl, haloalkyl, alkenyl, haloalkenyl, alkynyl, alkoxyalkyl, alkylthioalkyl or optionally substituted cycloalkyl or aryl, R³, R⁴ and R⁵ are, inter alia, H or alkyl and R⁶ is, inter alia, OH, are disclosed in EP0334133.

A problem that remains is the provision of alternative herbicidal pyrrolones.

A further problem that remains is the provision of herbicidal compounds having improved potency relative to known compounds.

A further problem that remains is the provision of herbicidal compounds having an improved spectrum of activity relative to known compounds.

A further problem that remains is the provision of herbicidal compounds having enhanced selectivity relative to known compounds.

These and other problems of the art are addressed by the present invention.

### SUMMARY OF THE INVENTION

In a first aspect, the invention provides compounds of the formula (I)

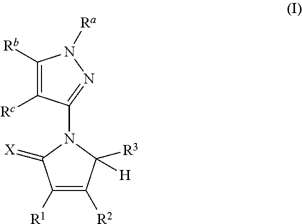

wherein
X is selected from S and O;
R^{a} is selected from hydrogen, C₁-C₆ alkyl and C₁-C₆ haloalkyl;
R^{b} is selected from hydrogen, formyl, hydroxyl, halogen, nitro, cyano, C₁-C₆ alkyl, C₁-C₆ cyanoalkyl, C₃-C₆ cycloalkyl, C₃-C₆ cyanocycloalkyl, C₁-C₆ haloalkyl, C₁-C₆ alkylthio, C₁-C₆ alkoxy, C₁-C₆ alkoxy C₁-C₆ alkyl, C₁-C₆ alkthio C₁-C₆ alkyl, C₁-C₆ cyanoalkoxy, C₁-C₆ haloalkoxy, C₁-C₆ alkoxy C₁-C₆ alkoxy, C₂-C₆ alkenyl, C₂-C₆ alkynyl, C₂-C₆ cyanoalkenyl, C₂-C₆ cyanoalkynyl, C₂-C₆ alkenyloxy, C₂-C₆ alkynyloxy, C₂-C₆ haloalkenyl, C₂-C₆ haloalkynyl, C₂-C₆ haloalkenyloxy, C₂-C₆ haloalkynyloxy, C₁-C₆ alkoxy C₂-C₆ alkenyl, C₁-C₆ alkoxy C₂-C₆ alkynyl, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylsulfonyloxy, C₁-C₆ alkylcarbonyl, C₁-C₆ haloalkylcarbonyl, C₂-C₆ alkenylcarbonyl, C₂-C₆ alkynylcarbonyl, C₂-C₆ haloalkenylcarbonyl, C₂-C₆ haloalkynylcarbonyl, tri C₁-C₆ alkylsilyl C₂-C₆ alkynyl, C₁-C₆ alkylamido, a group R⁵R⁶N—, a group R⁵C(O)N(R⁶)—, a group R⁵S(O₂)N(R⁶)—, a group R⁵R⁶NSO₂—, a C₆-C₁₀ aryl group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, a C₆-C₁₀ aryloxy group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, a C₆-C₁₀ benzyl group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, a C₆-C₁₀ benzyloxy group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, a C₃-C₆ heterocyclyl group optionally substituted by from 1 to 3 groups independently selected from C₁-C₄ alkyl and a C₃-C₆ cycloalkyl group optionally substituted with from 1 to 3 groups independently selected from halogen or C₁-C₆ alkyl;
R^{c} is selected from hydrogen, halogen, cyano, C₁-C₆ alkyl or C₁-C₆ haloalkyl;
or R^{a} and R^{b} together with the nitrogen and carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl;

or R^{b} and R^{c} together with the carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl;

R¹ is C₁-C₆ alkyl, C₁-C₆ haloalkyl or C₁-C₃ alkoxy and R² is halogen or C₁-C₃ alkoxy with the proviso that R¹ and R² are not both C₁-C₃ alkoxy;
R³ is selected from halogen, hydroxyl, or any one of the following groups

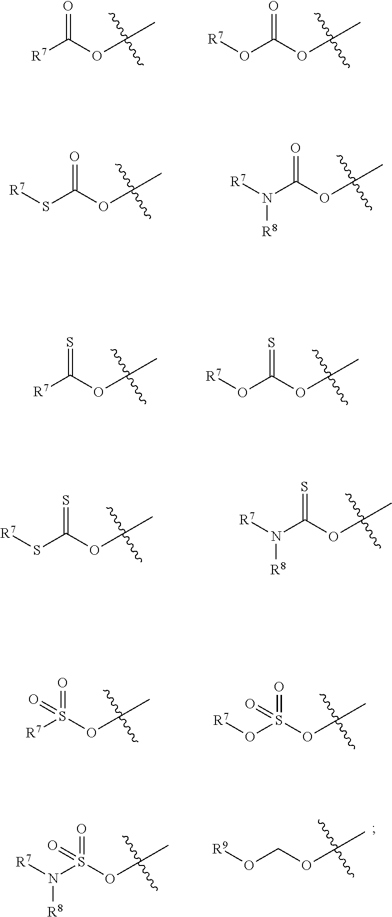

R⁵ and R⁶ are independently selected from hydrogen, C₁-C₆ alkyl, C₁-C₆ haloalkyl, C₂-C₆ alkenyl, C₂-C₆ alkynyl, or R⁵ and R⁶ together with the carbon atoms to which they are attached form a 3-6 membered saturated or partially unsaturated ring optionally comprising from 1 to 3 heteroatoms independently selected from S, O and N and optionally substituted with from 1 to 3 groups independently selected from halogen or C₁-C₆ alkyl;
R⁷ and R⁸ are independently selected from hydrogen, C₁-C₆ alkyl, C₁-C₆ haloalkyl, C₂-C₆ alkenyl, C₂-C₆ alkynyl, a C₅-C₁₀ heteroaryl group which can be mono- or bicyclic comprising from 1 to 4 heteroatoms independently selected from N, O and S and optionally substituted with 1 to 3 groups independently selected from halogen, C₁-C₃ alkyl, C₁-C₃ haloalkyl and C₁-C₃ alkoxy, a C₆-C₁₀ aryl group optionally substituted with 1 to 3 groups independently selected from halogen, nitro, cyano, C₁-C₃ alkyl, C₁-C₃ alkoxy, C₁-C₃ haloalkyl and C₁-C₃ haloalkoxy, or R⁷ and R⁸ together with the atoms to which they are attached form a 3-6 membered saturated or partially unsaturated ring optionally comprising from 1 to 3 heteroatoms independently selected from S, O and N and optionally substituted with from 1 to 3 groups independently selected from halogen or C₁-C₆ alkyl;
R⁹ is selected from C₁-C₆ alkyl or benzyl optionally substituted with 1 to 3 groups independently selected from halogen, nitro, cyano, C₁-C₃ alkyl, C₁-C₃ alkoxy, C₁-C₃ haloalkyl, and C₁-C₃ haloalkoxy;
or an N-oxide or salt form thereof.

In a second aspect, the invention provides herbicidal compositions comprising a compound of the invention together with at least one agriculturally acceptable adjuvant or diluent.

In a third aspect, the invention provides the use of a compound or a composition of the invention for use as a herbicide.

In a fourth aspect, the invention provides a method of controlling weeds in crops of useful plants, comprising applying to said weeds or to the locus of said weeds, or to said useful crop plants, a compound or a composition of the invention.

In a fifth aspect, the invention relates to processes useful in the preparation of compounds of the invention.

In a sixth aspect, the invention relates to intermediates useful in the preparation of compounds of the invention.

### DETAILED DESCRIPTION

In particularly preferred embodiments of the invention, the preferred groups for X, R¹, R², R³, R^{a}, R^{b} and R^{c}, in any combination thereof, are as set out below.

Preferably, X is O.

Preferably, R^{a} is selected from hydrogen, methyl, ethyl, C₁-C₂ haloalkyl or R^{a} and R^{b} together with the nitrogen and carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl. More preferably, R^{a} is selected from hydrogen, methyl, ethyl or R^{a} and R^{b} together with the nitrogen and carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl. Most preferably, R^{a} is selected from hydrogen or methyl or R^{a} and R^{b} together with the nitrogen and carbon atoms to which they are attached form a 5 or 6 membered saturated ring optionally substituted with 1 to 3 groups independently selected from C₁-C₃ alkyl.

Preferably, R^{b} is as defined above with the proviso that when R^{b} is C₂-C₆ alkynyl, C₂-C₆ cyanoalkynyl, C₂-C₆ haloalkynyl or C₁-C₆ alkoxy C₂-C₆ alkynyl, the alkynyl group is not directly attached to the pyrazole ring. More preferably, R^{b} is selected from hydrogen, halogen, cyano, C₁-C₆ alkyl, C₃-C₆ cycloalkyl, C₁-C₆ haloalkyl, C₂-C₆ alkenyl, C₁-C₆ cyanoalkyl, C₁-C₆ alkylthio, C₁-C₆ alkylsulfinyl, C₁-C₆ alkylsulphonyl, C₁-C₅ alkoxy C₁-C₆ alkyl, a C₆-C₁₀ aryl group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, a C₃-C₆ heteroaryl group optionally substituted by from 1 to 3 groups independently selected from C₁-C₄ alkyl, a C₆-C₁₀ benzyl group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, or R^{a} and R^{b} together with the nitrogen and carbon atoms to which they are attached form a 3-7 membered saturated or partially unsaturated ring optionally comprising 1 to 3 heteroatoms independently selected from S, O and N and optionally substituted with 1 to 3 groups independently selected from C₁-C₆ alkyl or R^{b} and R^{c} together with the carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl. Even more preferably, R^{b} is selected from hydrogen, halogen, C₁-C₆ alkyl, C₃-C₆ cycloalkyl, C₁-C₆ haloalkyl, C₂-C₆ alkenyl, a C₆-C₁₀ aryl group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, a C₃-C₆ heteroaryl group optionally substituted by from 1 to 3 groups independently selected from C₁-C₄ alkyl, a C₆-C₁₀ benzyl group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, or R^{a} and R^{b} together with the nitrogen and carbon atoms to which they are attached form a 3-7 membered saturated or partially unsaturated ring optionally comprising 1 to 3 heteroatoms independently selected from S, O and N and optionally substituted with 1 to 3 groups independently selected from C₁-C₆ alkyl or R^{b} and R^{c} together with the carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl. Even more preferably, R^{b} is selected from hydrogen, halogen, C₁-C₄ alkyl, C₁-C₄ haloalkyl, C₂-C₄ alkenyl or C₁-C₄ alkoxy or R^{a} and R^{b} together with the nitrogen and carbon atoms to which they are attached form a 3-7 membered saturated or partially unsaturated ring optionally comprising 1 to 3 heteroatoms independently selected from S, O and N and optionally substituted with 1 to 3 groups independently selected from C₁-C₆ alkyl, or R^{b} and R^{c} together with the carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl. Even more preferably, R^{b} is selected from halogen, C₁-C₄ alkyl or C₁-C₄ haloalkyl or R^{a} and R^{b} together with the nitrogen and carbon atoms to which they are attached form a 5 or 6 membered saturated ring optionally substituted with 1 to 3 groups independently selected from C₁-C₃ alkyl, or R^{b} and R^{c} together with the carbon atoms to which they are attached form a 5 or 6 membered saturated ring optionally substituted with from 1 to 3 groups independently selected from halogen, C₁-C₃ alkyl and C₁-C₃ haloalkyl. Even more preferably R^{b} is selected from bromo, chloro, fluoro, iso-propyl, tert-butyl or trifluoromethyl or R^{a} and R^{b} together with the nitrogen and carbon atoms to which they are attached form a 5 or 6 membered saturated ring optionally substituted with 1 to 3 groups independently selected from C₁-C₃ alkyl, or R^{b} and R^{c} together with the carbon atoms to which they are attached form a 5 or 6 membered saturated ring optionally substituted with from 1 to 3 groups independently selected from halogen, C₁-C₃ alkyl and C₁-C₃ haloalkyl. Most preferably, R^{b} is selected from iso-propyl, tert-butyl or trifluoromethyl or R^{a} and R^{b} together with the nitrogen and carbon atoms to which they are attached form a 5 or 6 membered saturated ring optionally substituted with 1 to 3 groups independently selected from C₁-C₃ alkyl, or R^{b} and R^{c} together with the carbon atoms to which they are attached form a 5 or 6 membered saturated ring optionally substituted with from 1 to 3 groups independently selected from halogen, C₁-C₃ alkyl and C₁-C₃ haloalkyl.

Preferably, R^{c} is selected from hydrogen, methyl, chloro or cyano or R^{b} and R^{c} together with the carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl. More preferably, R^{c} is selected from hydrogen, methyl or cyano or R^{b} and R^{c} together with the carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl. Most preferably, R^{c} is hydrogen or R^{b} and R^{c} together with the carbon atoms to which they are attached form a 5 or 6 membered saturated ring optionally substituted with from 1 to 3 groups independently selected from halogen, C₁-C₃ alkyl and C₁-C₃ haloalkyl.

In a preferred embodiment, the pyrazole ring is 5-tert-butyl-1-methyl-pyrazol-3-yl.

In a preferred embodiment, the pyrazole ring is 5-isopropyl-1-methyl-pyrazol-3-yl.

In a preferred embodiment, the pyrazole ring is 1-methyl-5-(trifluoromethyl)pyrazol-3-yl.

In a preferred embodiment, the pyrazole ring is 5-tert-butyl-1H-pyrazol-3-yl.

In a preferred embodiment, the pyrazole ring is 5-isopropyl-1H-pyrazol-3-yl.

In a preferred embodiment, the pyrazole ring is 5-(trifluoromethyl)-1H-pyrazol-3-yl.

In a preferred embodiment, the pyrazole ring is 5-(1,1-dimethylbut-3-enyl)-1-methyl-pyrazol-3-yl.

In a preferred embodiment, the pyrazole ring is 4,4-dimethyl-5,6-dihydropyrrolo[1,2-b]pyrazol-2-yl.

In a preferred embodiment, the pyrazole ring is 4,4-dimethyl-6,7-dihydro-5H-pyrazolo[1,5-a]pyridin-2-yl.

Preferably, R¹ is selected from methyl, ethyl, methoxy or ethoxy, with the proviso that when R¹ is methoxy or ethoxy, R² is not methoxy or ethoxy.

Preferably, R² is selected from bromo, chloro, methoxy or ethoxy, with the proviso that when R¹ is methoxy or ethoxy, R² is not methoxy or ethoxy.

More preferrably, (i) R¹ is methyl and R² is bromo, (ii) R¹ is methyl and R² is chloro, (iii) R¹ is methyl and R² is methoxy, (iv) R¹ is methoxy and R² is chloro or (v) R¹ is methoxy and R² is bromo.

Preferably, R³ is selected from halogen, hydroxyl, C₁-C₆ alkoxycarbonyloxy or aryloxycarbonyloxy wherein the aryl group may be substituted with 1 to 3 groups independently selected from halogen, nitro, cyano, C₁-C₃ alkyl, C₁-C₃ alkoxy, C₁-C₃ haloalkyl and C₁-C₃ haloalkoxy. More preferably, R³ is selected from hydroxyl or halogen. Most preferably, R³ is hydroxyl.

The compounds of formula (I) may exist as different geometric isomers, or in different tautomeric forms. This invention covers all such isomers and tautomers, and mixtures thereof in all proportions, as well as isotopic forms such as deuterated compounds. For example, compounds of formula (II) may exist in equilibrium with the tautomeric form (III).

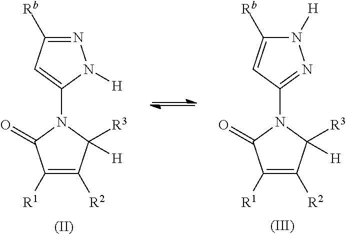

The compounds of this invention may contain an asymmetric carbon atom and some of the compounds of this invention may contain one or more asymmetric centers and may thus give rise to optical isomers and diastereomers. While shown without respect to stereochemistry, the present invention includes such optical isomers and diastereomers; as well as the racemic and resolved, enantiomerically pure R and S stereoisomers; as well as other mixtures of the R and S stereoisomers and agrochemically acceptable salts thereof. It is recognized that one optical isomer, including diastereomer and enantiomer, or stereoisomer may have favorable properties over the other. Thus when disclosing and claiming the invention, when one racemic mixture is disclosed, it is clearly contemplated that both optical isomers, including diastereomers and enantiomers, or stereoisomers substantially free of the other are disclosed and claimed as well.

Alkyl, as used herein refers to an aliphatic hydrocarbon chain and includes straight and branched chains e. g. of 1 to 6 carbon atoms such as methyl, ethyl, n-propyl, isopropyl, n-butyl, isobutyl, sec-butyl, t-butyl, n-pentyl, isopentyl, neo-pentyl, n-hexyl, and isohexyl.

Alkenyl, as used herein, refers to an aliphatic hydrocarbon chain having at least one double bond, and preferably one double bond, and includes straight and branched chains e. g. of 2 to 6 carbon atoms such as ethenyl (vinyl), prop-1-enyl, prop-2-enyl (allyl), isopropenyl, but-1-enyl, but-2-enyl, but-3-enyl, 2-methylpropenyl.

Alkynyl, as used herein, refers to an aliphatic hydrocarbon chain having at least one triple bond, and preferably one triple bond, and includes straight and branched chains e. g. of 2 to 6 carbon atoms such as ethynyl, prop-1-ynyl, prop-2-ynyl (propargyl) but-1-ynyl, but-2-ynyl and but-3-ynyl.

Cycloalkyl, as used herein, refers to a cyclic, saturated hydrocarbon group having from 3 to 6 ring carbon atoms. Examples of cycloalkyl groups are cyclopropyl, cyclobutyl, cyclopentyl and cyclohexyl.

Alkoxy as used herein refers to the group —OR, wherein R is alkyl as defined above. Examples of alkoxy groups include methoxy, ethoxy, n-propoxy, isopropoxy, n-butoxy, isobutoxy, sec-butoxy, t-butoxy, n-pentoxy, isopentoxy, neo-pentoxy, n-hexyloxy, and isohexyloxy.

Alkenyloxy refers to the group —OR, wherein R is alkenyl as defined above. Examples of alkenyloxy groups are ethenyloxy, propenyloxy, isopropenyloxy, but-1-enyloxy, but-2-enyloxy, but-3-enyloxy, 2-methypropenyloxy etc.

Alkynyloxy refers to the group —OR, wherein R is alkynyl is as defined above. Examples of alkynyloxy groups are ethynyloxy, propynyloxy, but-1-ynyloxy, but-2-ynyloxy and but-3-ynyloxy.

Alkoxyalkyl as used herein refers to the group —ROR, wherein each R is, independently, an alkyl group as defined above.

Alkoxyalkenyl as used herein refers to the group —ROR′, wherein R is an alkyl group as defined above and R′ is an alkenyl group as defined above.

Alkoxyalkynyl as used herein refers to the group —ROR′, wherein R is an alkyl group as defined above and R′ is an alkynyl group as defined above.

Alkoxyalkoxy, as used herein, refers to the group —OROR, wherein each R is, independently, an alkyl group as defined above.

Cyanoalkyl as used herein refers to an alkyl group substituted with one or more cyano groups.

Cyanoalkenyl as used herein refers to an alkenyl group substituted with one or more cyano groups.

Cyanoalkynyl as used herein refers to an alkynyl group substituted with one or more cyano groups.

Cyanocycloalkyl as used herein refers to an cycloalkyl group substituted with one or more cyano groups.

Cyanoalkoxy as used herein refers to the group —OR, wherein R is cyanoalkyl as defined above.

Halogen, halide and halo refer to iodine, bromine, chlorine and fluorine.

Haloalkyl as used herein refers to an alkyl group as defined above wherein at least one hydrogen atom has been replaced with a halogen atom as defined above. Examples of haloalkyl groups include chloromethyl, dichloromethyl, trichloromethyl, fluoromethyl, difluoromethyl and trifluoromethyl. Preferred haloalkyl groups are fluoroalkyl groups {i.e. haloalkyl groups, containing fluorine as the only halogen). More highly preferred haloalkyl groups are perfluoroalkyl groups, i.e. alkyl groups wherein all the hydrogen atoms are replaced with fluorine atoms.

Haloalkenyl as used herein refers to an alkenyl group as defined above wherein at least one hydrogen atom has been replaced with a halogen atom as defined above.

Haloalkynyl as used herein refers to an alkynyl group as defined above wherein at least one hydrogen atom has been replaced with a halogen atom as defined above.

Haloalkoxy as used herein refers to the group —OR, wherein R is haloalkyl as defined above.

Haloalkenyloxy as used herein refers to the group —OR, wherein R is haloalkenyl as defined above.

Haloalkynyloxy as used herein refers to the group —OR, wherein R is haloalkynyl as defined above.

Alkylthio as used herein refers to the group —SR, wherein R is an alkyl group as defined above. Alkylthio groups include, but are not limited to, methylthio, ethylthio, propylthio, tert-butylthio, and the like.

Alkylthioalkyl as used herein refers to the group —RSR, wherein each R is, independently, an alkyl group as defined above.

Haloalkylthio as used herein refers to the group —SR, wherein R is a haloalkyl group as defined above.

Alkylsulfinyl as used herein refers to the group —S(O)R, wherein R is an alkyl group as defined above.

Alkylsulfonyl as used herein refers to the group —S(O)₂R, wherein R is an alkyl group as defined above.

Haloalkylsulfinyl as used herein refers to the group —S(O)R, wherein R is a haloalkyl group as defined above.

Haloalkylsulfonyl as used herein refers to the group —S(O)₂R, wherein R is a haloalkyl group as defined above.

Alkylsulfonyloxy, as used herein refers to the group —OSO₂R, wherein R is an alkyl group as defined above.

Alkylcarbonyl, as used herein refers to the group —COR, wherein R is an alkyl group as defined above. Examples of alkylcarbonyl groups include ethanoyl, propanoyl, n-butanoyl, etc.

Alkenylcarbonyl, as used herein refers to the group —COR, wherein R is an alkenyl group as defined above.

Alkynylcarbonyl, as used herein refers to the group —COR, wherein R is an alkynyl group as defined above.

Haloalkylcarbonyl, as used herein refers to the group —COR, wherein R is a haloalkyl group as defined above.

Haloalkenylcarbonyl, as used herein refers to the group —COR, wherein R is a haloalkenyl group as defined above.

Haloalkynylcarbonyl, as used herein refers to the group —COR, wherein R is a haloalkynyl group as defined above.

Alkoxycarbonyloxy as used herein, refers to the group —OC(O)OR, wherein R is an alkyl group as defined above. Examples of alkoxycarbonyloxy groups are methoxycarbonyloxy, ethoxycarbonyloxy, propoxycarbonyloxy, but-1-oxycarbonyloxy, but-2-oxycarbonyloxy and but-3-oxycarbonyloxy.

Trialkylsilylalkynyl, as used herein, refers to the group —RSi(R′)₃, wherein R is an alkynyl group as defined above and each R′ is, independently, selected from an alkyl group as defined above.

Formyl, as used herein, refers to the group —C(O)H.

Hydroxy or hydroxyl, as used herein, refers to the group —OH.

Nitro, as used herein, refers to the group —NO₂.

Cyano as used herein, refers to the group —CN.

Aryl, as used herein, refers to an unsaturated aromatic carbocyclic group of from 6 to 10 carbon atoms having a single ring (e. g., phenyl) or multiple condensed (fused) rings, at least one of which is aromatic (e.g., indanyl, naphthyl). Preferred aryl groups include phenyl, naphthyl and the like. Most preferably, an aryl group is a phenyl group.

Aryloxy, as used herein, refers to the group —O-aryl, wherein aryl is as defined above. Preferred aryloxy groups include phenoxy, naphthyloxy and the like.

Aryloxycarbonyloxy as used herein, refers to the group —OC(O)O-aryl wherein aryl is a as defined above.

Benzyl, as used herein, refers to the group —CH₂C₆H₅. Benzyl groups may be substituted on the alkyl linker or on the ring.

Benzyloxy, as used herein, refers to the group —OCH₂C₆H₅. Benzyloxy groups may be substituted on the linker or on the ring.

Heterocyclyl, as used herein, refers to a non-aromatic ring system containing 3 to 10 ring atoms, at least one ring heteroatom and consisting either of a single ring or of two or more fused rings. Preferably, single rings will contain up to three and bicyclic systems up to four heteroatoms which will preferably be chosen from nitrogen, oxygen and sulfur. When a ring system contains a sulphur atom, the sulphur atom may be present in any one of its oxidation states e.g. —S—, —S(═O)— or —S(═O₂)—. Examples of such groups include pyrrolidinyl, imidazolinyl, pyrazolidinyl, piperidyl, piperazinyl, quinuclidinyl, morpholinyl, together with unsaturated or partially unsaturated analogues such as 4,5,6,7-tetrahydro-benzothiophenyl, chromen-4-onyl, 9H-fluorenyl, 3,4-dihydro-2H-benzo-1,4-dioxepinyl, 2,3-dihydro-benzofuranyl, piperidinyl, 1,3-dioxolanyl, 1,3-dioxanyl, 4,5-dihydro-isoxazolyl, tetrahydrofuranyl and morpholinyl.

Heteroaryl, as used herein, refers to a ring system containing 5 to 10 ring atoms, 1 to 4 ring heteroatoms and consisting either of a single aromatic ring or of two or more fused rings, at least one of which is aromatic. Preferably, single rings will contain up to three and bicyclic systems up to four heteroatoms which will preferably be independently chosen from nitrogen, oxygen and sulfur. When a ring system contains a sulphur atom, the sulphur atom may be present in any one of its oxidation states e.g. —S—, —S(═O)— or —S(═O₂)—. Examples of such groups include pyridyl, pyridazinyl, pyrimidinyl, pyrazinyl, triazinyl, furanyl, thiophenyl, oxazolyl, isoxazolyl, oxadiazolyl, thiazolyl, isothiazolyl, thiadiazolyl, pyrrolyl, pyrazolyl, imidazolyl, triazolyl and tetrazolyl. Examples of bicyclic groups are benzothiophenyl, benzimidazolyl, benzothiadiazolyl, quinolinyl, cinnolinyl, quinoxalinyl and pyrazolo[1,5-a]pyrimidinyl.

‘Saturated ring’, as used herein, refers to a ring system in which the atoms in the ring are linked by single bonds and may consist of either a single ring or two or more fused rings.

‘Partially unsaturated ring’, as used herein, refers to a ring system in which at least two atoms in the ring are linked by a double bond and may consist of either a single ring or two or more fused rings. Partially unsaturated ring systems do not include aromatic rings.

“Optionally substituted” as used herein means the group referred to can be substituted at one or more positions by any one or any combination of the radicals listed thereafter. For most groups, one or more hydrogen atoms are replaced by the radicals listed thereafter. For halogenated groups, for example, haloalkyl groups, one or more halogen atoms are replaced by the radicals listed thereafter.

Suitable salts include those derived from alkali or alkaline earth metals and those derived from ammonia and amines. Preferred cations include sodium, potassium, magnesium, and ammonium cations of the formula N⁺(R¹⁹R²⁰R²¹R²²), wherein R¹⁹, R²⁰, R²¹ and R²² are independently selected from hydrogen, C₁-C₆ alkyl and C₁-C₆ hydroxyalkyl. Salts of the compounds of Formula I can be prepared by treatment of compounds of Formula I with a metal hydroxide, such as sodium hydroxide, or an amine, such as ammonia, trimethylamine, diethanolamine, 2-methylthiopropylamine, bisallylamine, 2-butoxyethylamine, morpholine, cyclododecylamine, or benzylamine. Amine salts are often preferred forms of the compounds of Formula I because they are water-soluble and lend themselves to the preparation of desirable aqueous based herbicidal compositions.

Acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids when a compound of this invention contains a basic moiety.

In another aspect the present invention provides intermediates useful in the preparation of compounds of the invention.

In one embodiment, there are provided intermediates of formula (IV)

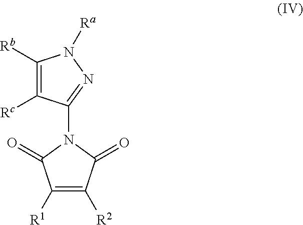

wherein R¹, R², R^{a}, R^{b} and R^{c} are as defined above.

Compounds of the invention may be prepared by techniques known to the person skilled in the art of organic chemistry. General methods for the production of compounds of formula (I) are described below. Unless otherwise stated in the text, the substituents X, A, R¹, R², R³, R^{a}, R^{b} and R^{c} are as defined hereinbefore. The starting materials used for the preparation of the compounds of the invention may be purchased from usual commercial suppliers or may be prepared by known methods. The starting materials as well as the intermediates may be purified before use in the next step by state of the art methodologies such as chromatography, crystallization, distillation and filtration.

For example, compounds of formula (I) wherein R³ is a hydroxyl group may be prepared by reaction of substituted maleic anhydride (V) with amine (VI) in acetic acid to give maleimide (IV), and subsequent reduction with e.g. sodium borohydride to give compound (VII) (compound (I) wherein R³ is hydroxyl), together with regioisomer (VIII) as a side-product (Scheme 1). Suitable conditions for achieving these transformations are disclosed in CH633678. Maleic anhydrides (IV) can be prepared by literature methods (Journal of the Chemical Society, Perkin Transactions 1: Organic and Bio-Organic Chemistry (1972-1999), 1982, p. 215-222, EP1426365 A1, 2004, Journal of Organic Chemistry, 1998, vol. 63, 8, p. 2646-2655).

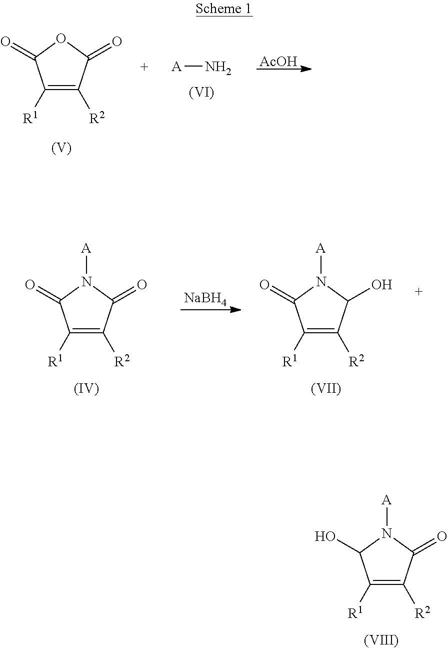

wherein A is an optionally substituted pyrazole ring.

Alternatively compounds of formula (I) wherein R³ is a hydroxyl group may be prepared by reaction of bromolactone (IX) with the appropriate amino pyrazole (VI), in a solvent, such as toluene with a suitable base, such as triethylamine to afford intermediate (X). Heating (X) in acetic anhydride and pyridine affords (XI). Heating (XI) in an acetic acid/water solution affords the desired final compounds (XII) (Scheme 2).

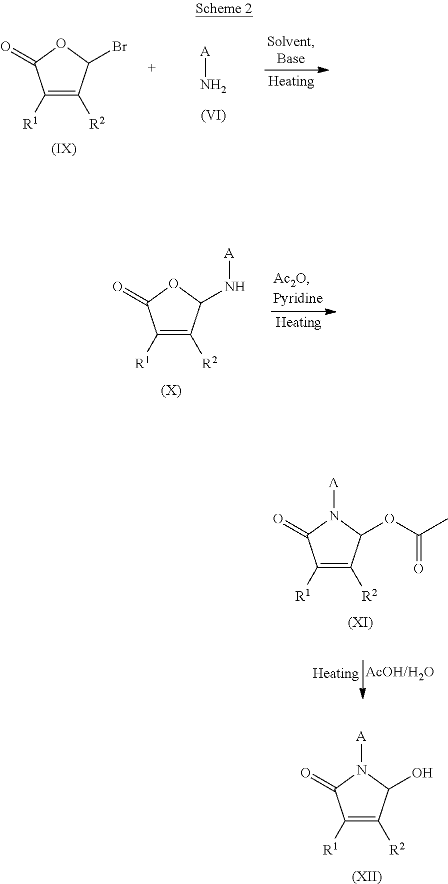

The relevant amino pyrazoles can be prepared as shown in Schemes 3 to 12.

Reaction of hydrazine, or an appropriate salt, with a β-ketonitrile in a solvent such as ethanol affords the desired amino pyrazoles (VI) where R^{a} and R^{c}═H (Scheme 3) as described in Journal of Medicinal Chemistry, 2008, vol. 51, No. 15 p. 4672-4684.

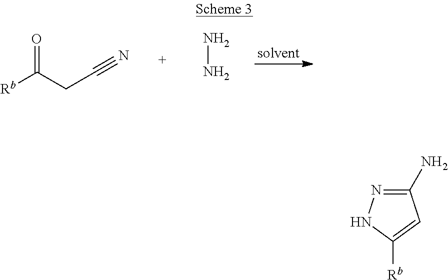

Alternatively, reaction of an alkyl hydrazine, or an appropriate salt, with a nitrile vinyl chloride (XIII), or its isomers (XIII), in a solvent such as ethanol, with an appropriate base, such as K₂CO₃, affords the desired amino pyrazoles (XIV) and undesired isomer (XV) (Scheme 4) as described in Pharmazie, 1989, vol. 44, No. 8 p. 535-539 or Journal of Heterocyclic Chemistry, 1982, vol. 19, p. 1267-1273.

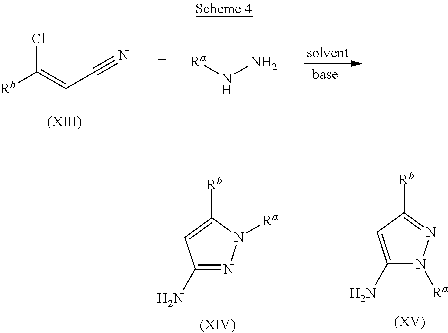

Nitrile vinyl chlorides (XIII) can be prepared from the corresponding β-ketonitrile and a suitable chlorination reagent such as PCl₅ or POCl₃, in a suitable solvent, such as dichlormethane as shown in Scheme 5. Alternatively the nitrile vinyl chlorides (XIII) can be prepared from the corresponding ketone (Scheme 5).

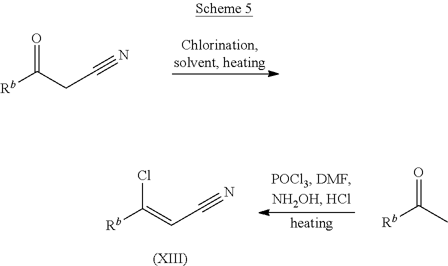

Alternatively nitrile vinyl chlorides (XVII) can be prepared from the corresponding aldehyde and phosphonate (XVI), with an appropriate base, such as LiN(TMS)₂ in an appropriate solvent, such as THF (Scheme 6). Phosphonate (XVI) can be prepared as described in J. Chem. Soc., Perkin Trans. 1, 2000, 3311-3316.

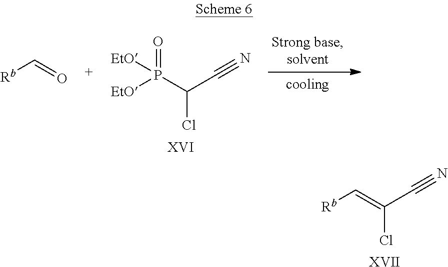

Alternatively the amino pyrazoles can be prepared from the corresponding pyrazole 3-carboxylates (XVIII). N-alkylation employing an appropriate base, such as tBuOK, in the appropriate solvent, such as THF, with the relevant alkyl halide, followed by ester hydrolysis affords the pyrazole 3-carboxylic acids (XIX). Reaction of (XIX) with DPPA in a solvent, such as tBuOH, and triethylamine affords a mixture of the urea (XX) and the desired amino pyrazole (XXI). (XX) may be further converted into (XXI) under hydrolysis conditions (Scheme 7).

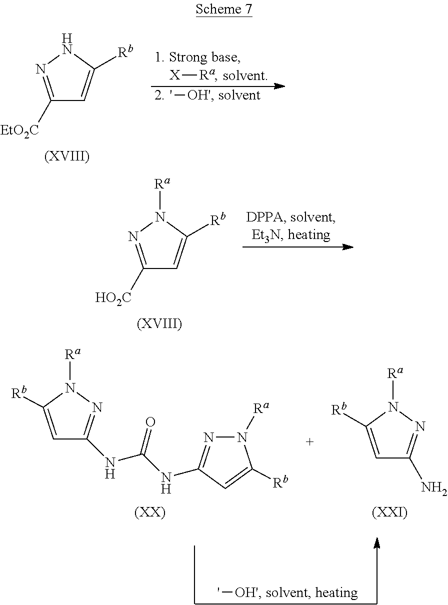

Alternatively deprotonation of pyrazole (XXII) with an appropriate base, such as BuLi, followed by quenching with an electrophile, such as halogens, alkyl halides, aldehydes, ketones etc as described in Journal of Organic Chemistry, 1984, vol. 49, No. 7 p. 1224-1227, affords (XXIII). Deprotection of the pyrrole masked amine (XXIII), also described in the above reference, affords the desired amino pyrazoles (XXI). Alternatively, further functional group transformations of (XXIII), which will be know to those skilled in the art, can be used to further vary the pyrazole 5-position (R^{b}) before deprotection to (XXI) (Scheme 8).

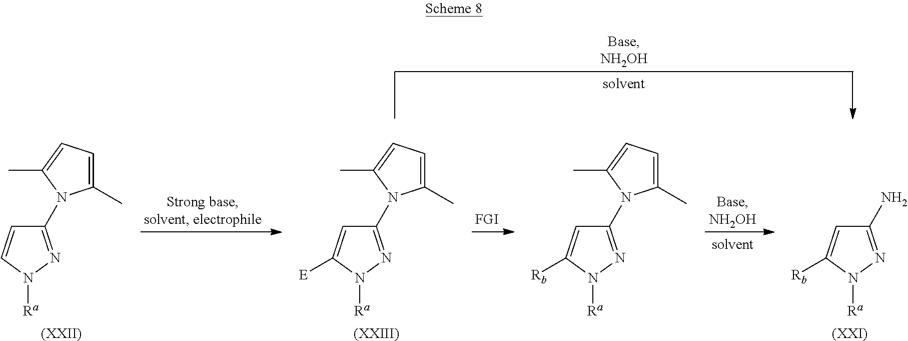

Phthalimide protected pyrazoles (XXIV) can be akylated with an appropriate base such as K₂CO₃, tBuOK, NaH, NaOH, in an appropriate solvent, such as THF and ether, with the appropriate alkyl halide, to afford (XXV) and varying amounts of undesired (XXVI). Removal of the protecting group, employing, propane-1,2-diamine affords the the desired amino pyrazoles (XXI) (Scheme 9).

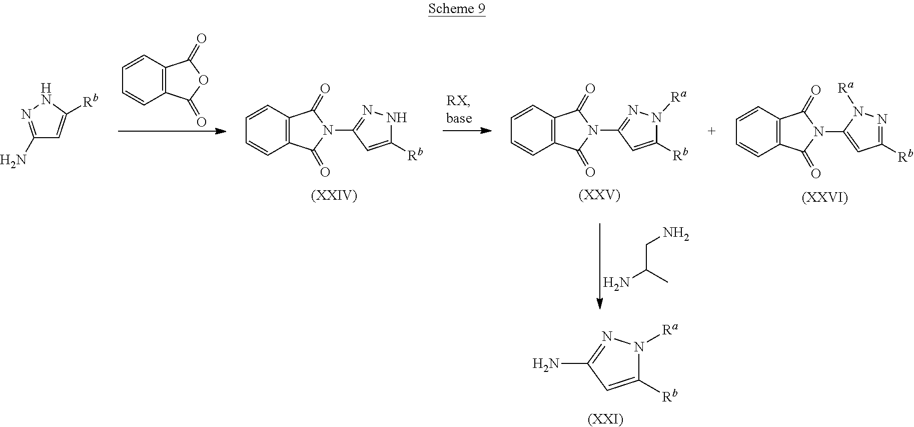

Fused bycyclic amino pyrazoles of type (XXIX) can be prepared from Lactam intermediates (XXVIII) as shown in Scheme 10, wherein R¹⁰ and R¹¹ are, for example, H or C₁-C₆ alkyl.

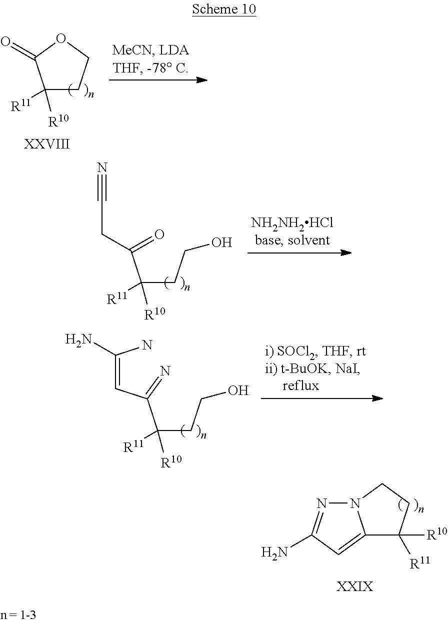

Fused bycyclic amino pyrazoles of type (XXXI) can be prepared from cyclic ketone intermediates intermediates (XXX) as shown in Scheme 11.

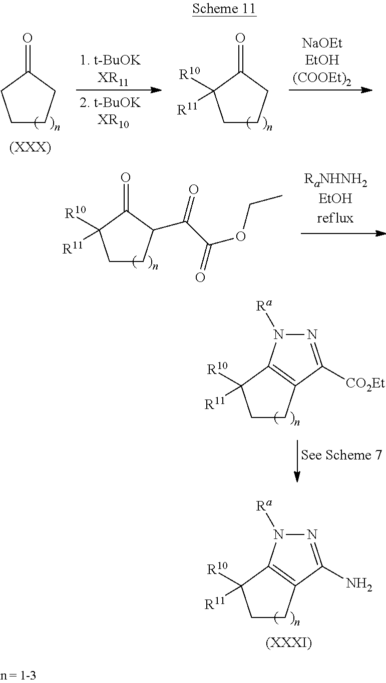

3-amino-4-nitrile substituted pyrazoles may be prepared as shown in Scheme 12 as reported in the literature. Journal of Heterocyclic Chemistry, 1982, vol. 19, p. 1267-1273.

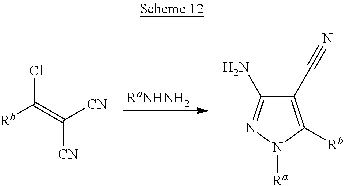

Compound (XXXII) may be halogenated (i), alkylated (ii), acylated (iii), sulfonylated (iv) or alkoxyacylated (v), under standard conditions to access other compounds having different values of R³ (Scheme 13).

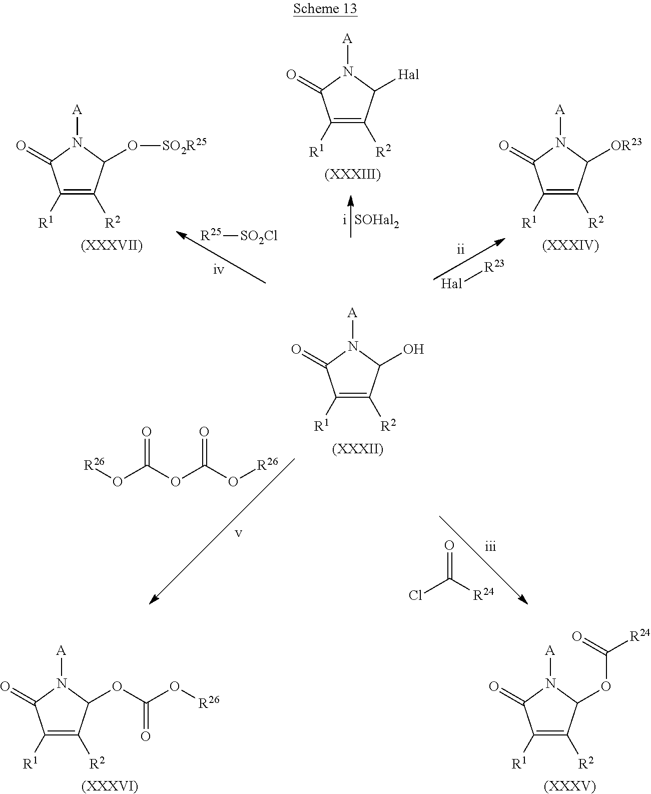

wherein R¹ and R² areas defined above, A is an optionally substituted pyrazole ring, Hal is halogen as defined above, R²³ is selected from C₁-C₆ alkyl, C₂-C₆ alkenyl, C₂-C₆ alkynyl, R²⁴ is selected from H and C₁-C₅ alkyl, R²⁵ is selected from C₁-C₆ alkyl and C₆-C₁₀ aryl optionally substituted with 1 to 3 groups independently selected from halogen, C₁-C₃ alkyl, and C₁-C₃ alkoxy and R²⁶ is selected from C₁-C₅ alkyl.

Suitable conditions for effecting transformations i to v will be known to those skilled in the art, and are set out for example in J. March, Advanced Organic Chemistry, 4th ed. Wiley, New York, 1992, and references cited therein.

Compounds of formula (VII) wherein R¹ is alkoxy and R² is a halogen may be prepared by reaction of substituted pyrazole amine (VI), formaldehyde and an α-haloketo acid, in a suitable solvent and optionally in the presence of acid to give 2-hydroxy lactam (XXXVIII). Similar processes are described, e.g. in Clarke et al., JACS, 1933, 55, 4571-4587). Subsequent alkylation followed by treated with suitable oxidants lead to the desired products (VII) directly, or can be converted to compounds (XXXIX), e.g. by treating with manganese triacetate in glacial acetic acid followed by hydrolysis to give compounds of formula (VII) (Scheme 14).

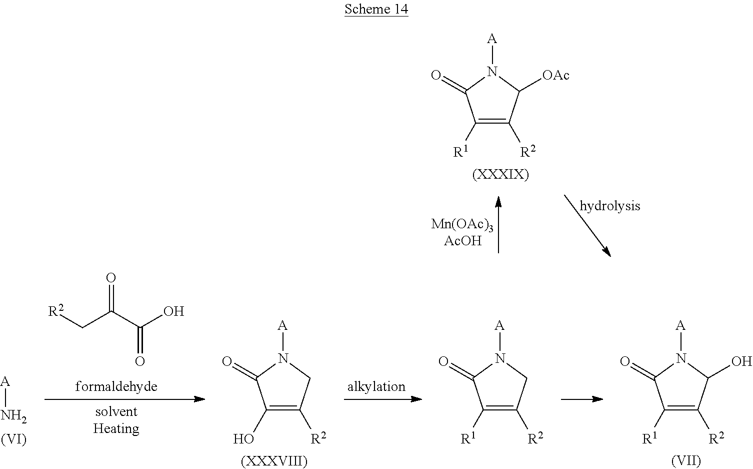

The compounds of formula (I) according to the invention can be used as herbicides in unmodified form, as obtained in the synthesis, but they are generally formulated into herbicidal compositions in various ways using formulation adjuvants, such as carriers, solvents and surface-active substances. Therefore, the invention also relates to a herbicidal composition which comprises a herbicidally effective amount of a compound of formula (I) in addition to formulation adjuvants. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO Specifications for Plant Protection Products, 5th Edition, 1999. Such formulations can either be used directly or they are diluted prior to use. The dilutions can be made, for example, with water, liquid fertilizers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof. The active ingredients can also be contained in very fine microcapsules consisting of a polymer. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art in this connection. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol (PEG400), propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydro-furfuryl alcohol, hexanol, octanol, ethylene glycol, propylene glycol, glycerol, N-methyl-2-pyrrolidone and the like. Water is generally the carrier of choice for diluting the concentrates. Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances, as described, for example, in CFR 180.1001. (c) & (d).

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecyl-benzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in “McCutcheon's Detergents and Emulsifiers Annual” MC Publishing Corp., Ridgewood N.J., 1981.

Further adjuvants that can usually be used in pesticidal formulations include crystallization inhibitors, viscosity modifiers, suspending agents, dyes, anti-oxidants, foaming agents, light absorbers, mixing auxiliaries, antifoams, complexing agents, neutralizing or pH-modifying substances and buffers, corrosion inhibitors, fragrances, wetting agents, take-up enhancers, micronutrients, plasticisers, glidants, lubricants, dispersants, thickeners, antifreezes, microbicides, and also liquid and solid fertilizers.

The compositions according to the invention can additionally include an additive comprising an oil of vegetable or animal origin, a mineral oil, alkyl esters of such oils or mixtures of such oils and oil derivatives. The amount of oil additive in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil, olive oil or sunflower oil, emulsified vegetable oil, such as AMIGO® (Rhone-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈-C₂₂ fatty acids, especially the methyl derivatives of C₁₂-C₁₈ fatty acids, for example the methyl esters of lauric acid, palmitic acid and oleic acid, being of importance. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000.

The application and action of the oil additives can be further improved by combination with surface-active substances, such as non-ionic, anionic or cationic surfactants. Examples of suitable anionic, non-ionic and cationic surfactants are listed on pages 7 and 8 of WO 97/34485. Preferred surface-active substances are anionic surfactants of the dodecylbenzylsulfonate type, especially the calcium salts thereof, and also non-ionic surfactants of the fatty alcohol ethoxylate type. Special preference is given to ethoxylated C₁₂-C₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltriloxanes which are commercially available e.g. as Silwet L-77®, and also perfluorinated surfactants. The concentration of the surface-active substances in relation to the total additive is generally from 1 to 30% by weight. Examples of oil additives consisting of mixtures of oil or mineral oils or derivatives thereof with surfactants are Edenor ME SU®, Turbocharge® (Syngenta AG, CH) or ActipronC (BP Oil UK Limited, GB).

If desired, it is also possible for the mentioned surface-active substances to be used in the formulations on their own, that is to say, without oil additives.

Furthermore, the addition of an organic solvent to the oil additive/surfactant mixture may contribute to an additional enhancement of action. Suitable solvents are, for example, Solvesso® (ESSO) or Aromatic Solvent® (Exxon Corporation). The concentration of such solvents can be from 10 to 80% by weight of the total weight. Oil additives that are present in admixture with solvents are described, for example, in U.S. Pat. No. 4,834,908. A commercially available oil additive disclosed therein is known by the name MERGE® (BASF Corporation). A further oil additive that is preferred according to the invention is SCORE® (Syngenta Crop Protection Canada).

In addition to the oil additives listed above, for the purpose of enhancing the action of the compositions according to the invention it is also possible for formulations of alkylpyrrolidones (e.g. Agrimax®) to be added to the spray mixture. Formulations of synthetic lattices, e.g. polyacrylamide, polyvinyl compounds or poly-1-p-menthene (e.g. Bond®, Courier® or Emerald®) may also be used. It is also possible for solutions that contain propionic acid, for example Eurogkem Pen-e-trate®, to be added to the spray mixture as action-enhancing agent.

The herbicidal compositions generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. Whereas commercial products will preferably be formulated as concentrates, the end user will normally employ dilute formulations.

The rates of application of compounds of formula (I) may vary within wide limits and depend on the nature of the soil, the method of application (pre- or post-emergence; seed dressing; application to the seed furrow; no tillage application etc.), the crop plant, the grass or weed to be controlled, the prevailing climatic conditions, and other factors governed by the method of application, the time of application and the target crop. The compounds of formula (I) according to the invention are generally applied at a rate of from 10 to 2000 g/ha, especially from 50 to 1000 g/ha.

Preferred formulations have especially the following compositions (%=percent by weight):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The following Examples further illustrate, but do not limit, the invention.

### Formulation Examples for Herbicides of Formula (I) (%=% by Weight)

| F1. Emulsifiable concentrates | a) | b) | c) | d) |
|---|---|---|---|---|
| active ingredient | 5% | 10% | 25% | 50% |
| calcium dodecylbenzenesulfonate | 6% | 8% | 6% | 8% |
| castor oil polyglycol ether | 4% | — | 4% | 4% |
| (36 mol of ethylene oxide) | | | | |
| octylphenol polyglycol ether | — | 4% | — | 2% |
| (7-8 mol of ethylene oxide) | | | | |
| NMP | — | — | 10% | 20% |
| arom. hydrocarbon mixture | 85% | 78% | 55% | 16% |
| C₉-C₁₂ | | | | |

Emulsions of any desired concentration can be obtained from such concentrates by dilution with water.

| F2. Solutions | a) | b) | c) | d) |
|---|---|---|---|---|
| active ingredient | 5% | 10% | 50% | 90% |
| 1-methoxy-3-(3-methoxy- | — | 20% | 20% | — |
| propoxy)-propane | | | | |
| polyethylene glycol MW 400 | 20% | 10% | — | — |
| NMP | — | — | 30% | 10% |
| arom. hydrocarbon mixture | 75% | 60% | — | — |
| C₉-C₁₂ | | | | |

The solutions are suitable for use in the form of microdrops.

| F3. Wettable powders | a) | b) | c) | d) |
|---|---|---|---|---|
| active ingredient | 5% | 25% | 50% | 80% |
| sodium lignosulfonate | 4% | — | 3% | — |
| sodium lauryl sulfate | 2% | 3% | — | 4% |
| sodium diisobutylnaphthalene- | — | 6% | 5% | 6% |
| sulfonate | | | | |
| octylphenol polyglycol ether | — | 1% | 2% | — |
| (7-8 mol of ethylene oxide) | | | | |
| highly dispersed silicic acid | 1% | 3% | 5% | 10% |
| kaolin | 88% | 62% | 35% | — |

The active ingredient is mixed thoroughly with the adjuvants and the mixture is thoroughly ground in a suitable mill, affording wettable powders which can be diluted with water to give suspensions of any desired concentration.

| F4. Coated granules | a) | b) | c) |
|---|---|---|---|
| active ingredient | 0.1% | 5% | 15% |
| highly dispersed silicic acid | 0.9% | 2% | 2% |
| inorganic carrier | 99.0% | 93% | 83% |
| (diameter 0.1-1 mm) | | | |
| e.g. CaCO₃ or SiO₂ | | | |

The active ingredient is dissolved in methylene chloride and applied to the carrier by spraying, and the solvent is then evaporated off in vacuo.

| F5. Coated granules | a) | b) | c) |
|---|---|---|---|
| active ingredient | 0.1% | 5% | 15% |
| polyethylene glycol MW 200 | 1.0% | 2% | 3% |
| highly dispersed silicic acid | 0.9% | 1% | 2% |
| inorganic carrier | 98.0% | 92% | 80% |
| (diameter 0.1-1 mm) | | | |
| e.g. CaCO₃ or SiO₂ | | | |

The finely ground active ingredient is uniformly applied, in a mixer, to the carrier moistened with polyethylene glycol. Non-dusty coated granules are obtained in this manner.

| F6. Extruder granules | a) | b) | c) | d) |
|---|---|---|---|---|
| active ingredient | 0.1% | 3% | 5% | 15% |
| sodium lignosulfonate | 1.5% | 2% | 3% | 4% |
| carboxymethylcellulose | 1.4% | 2% | 2% | 2% |
| kaolin | 97.0% | 93% | 90% | 79% |

The active ingredient is mixed and ground with the adjuvants, and the mixture is moistened with water. The mixture is extruded and then dried in a stream of air.

| F7. Dusts | a) | b) | c) |
|---|---|---|---|
| active ingredient | 0.1% | 1% | 5% |
| talcum | 39.9% | 49% | 35% |
| kaolin | 60.0% | 50% | 60% |

Ready-to-use dusts are obtained by mixing the active ingredient with the carriers and grinding the mixture in a suitable mill.

| F8. Suspension concentrates | a) | b) | c) | d) |
|---|---|---|---|---|
| active ingredient | 3% | 10% | 25% | 50% |
| ethylene glycol | 5% | 5% | 5% | 5% |
| nonylphenol polyglycol ether | — | 1% | 2% | — |
| (15 mol of ethylene oxide) | | | | |
| sodium lignosulfonate | 3% | 3% | 4% | 5% |
| carboxymethylcellulose | 1% | 1% | 1% | 1% |
| 37% aqueous formaldehyde | 0.2% | 0.2% | 0.2% | 0.2% |
| solution | | | | |
| silicone oil emulsion | 0.8% | 0.8% | 0.8% | 0.8% |
| water | 87% | 79% | 62% | 38% |

The finely ground active ingredient is intimately mixed with the adjuvants, giving a suspension concentrate from which suspensions of any desired concentration can be obtained by dilution with water.

The invention also provides a method of controlling plants which comprises applying to the plants or to the locus thereof a herbicidally effective amount of a compound of formula (I).

The invention also provides a method of inhibiting plant growth which comprises applying to the plants or to the locus thereof a herbicidally effective amount of a compound of formula (I).

The invention also provides a method of controlling weeds in crops of useful plants, comprising applying to said weeds or to the locus of said weeds, or to said useful plants or to the locus of said useful plants, a compound or a composition of the invention. Preferably, said crop of useful plants is a crop of maize plants.

The invention also provides a method of selectively controlling grasses and/or weeds in crops of useful plants which comprises applying to the useful plants or locus thereof or to the area of cultivation a herbicidally effective amount of a compound of formula (I). Preferably, said crop of useful plants is a crop of maize plants.

The term “herbicide” as used herein means a compound that controls or modifies the growth of plants. The term “herbicidally effective amount” means the quantity of such a compound or combination of such compounds that is capable of producing a controlling or modifying effect on the growth of plants. Controlling or modifying effects include all deviation from natural development, for example: killing, retardation, leaf burn, albinism, dwarfing and the like. The term “plants” refers to all physical parts of a plant, including seeds, seedlings, saplings, roots, tubers, stems, stalks, foliage, and fruits. The term “locus” is intended to include soil, seeds, and seedlings, as well as established vegetation and includes not only areas where weeds may already be growing, but also areas where weeds have yet to emerge, and also to areas under cultivation with respect to crops of useful plants. “Areas under cultivation” include land on which the crop plants are already growing and land intended for cultivation with such crop plants. The term “weeds” as used herein means any undesired plant, and thus includes not only agronomically important weeds as described below, but also volunteer crop plants.

The compounds of the invention can be applied before or after planting of the crops, before weeds emerge (pre-emergence application) or after weeds emerge (post-emergence application), and are particularly effective when applied post-emergence to the weeds.

Crops of useful plants in which the composition according to the invention can be used include, but are not limited to, perennial crops, such as citrus fruit, grapevines, nuts, oil palms, olives, pome fruit, stone fruit and rubber, and annual arable crops, such as cereals, for example barley and wheat, cotton, oilseed rape, maize, rice, soy beans, sugar beet, sugar cane, sunflowers, ornamentals, switchgrass, turf and vegetables, especially cereals, maize and soy beans.

The grasses and weeds to be controlled may be both monocotyledonous species, for example Agrostis, Alopecurus, Avena, Brachiaria, Bromus, Cenchrus, Cyperus, Digitaria, Echinochloa, Eriochloa, Lolium, Monochoria, Panicum, Poa, Rottboellia, Sagittaria, Scirpus, Setaria, Sida and Sorghum, and dicotyledonous species, for example Abutilon, Amaranthus, Chenopodium, Chrysanthemum, Euphorbia, Galium, Ipomoea, Kochia, Nasturtium, Polygonum, Sida, Sinapis, Solanum, Stellaria, Veronica, Viola and Xanthium.

Crops are to be understood as also including those crops which have been rendered tolerant to herbicides or classes of herbicides (e.g. auxins or ALS-, EPSPS-, PPO- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®, respectively.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesize such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood as being those which are obtained by conventional methods of breeding or genetic engineering and contain so-called output traits (e.g. improved storage stability, higher nutritional value and improved flavor).

Any method of application to weeds/crop of useful plant, or locus thereof, which is routinely used in agriculture may be used, for example application by spray or broadcast method typically after suitable dilution of a compound of formula (I) (whether said compound is formulated and/or in combination with one or more further active ingredients and/or safeners, as described herein).

The compounds of formula (I) according to the invention can also be used in combination with other active ingredients, e.g. other herbicides, and/or insecticides, and/or acaricides, and/or nematocides, and/or molluscicides, and/or fungicides, and/or plant growth regulators. Such mixtures, and the use of such mixtures to control weeds and/or undesired plant growth, form yet further aspects of the invention. For the avoidance of doubt, mixtures of invention also include mixtures of two or more different compounds of formula (I). In particular, the present invention also relates to a composition of the invention which comprises at least one further herbicide in addition to the compound of formula (I).

When a compound of formula (I) is combined with at least one additional herbicide, the following mixtures of the compound of formula (I) are preferred. Compound of formula (I)+acetochlor, compound of formula (I)+acifluorfen, compound of formula (I)+acifluorfen-sodium, compound of formula (I)+aclonifen, compound of formula (I)+acrolein, compound of formula (I)+alachlor, compound of formula (I)+alloxydim, compound of formula (I)+allyl alcohol, compound of formula (I)+ametryn, compound of formula (I)+amicarbazone, compound of formula (I)+amidosulfuron, compound of formula (I)+aminocyclopyrachlor, compound of formula (I)+aminopyralid, compound of formula (I)+amitrole, compound of formula (I)+ammonium sulfamate, compound of formula (I)+anilofos, compound of formula (I)+asulam, compound of formula (I)+atrazine, formula (I)+aviglycine, formula (I)+azafenidin, compound of formula (I)+azimsulfuron, compound of formula (I)+BCPC, compound of formula (I)+beflubutamid, compound of formula (I)+benazolin, formula (I)+bencarbazone, compound of formula (I)+benfluralin, compound of formula (I)+benfuresate, compound of formula (I)+bensulfuron, compound of formula (I)+bensulfuron-methyl, compound of formula (I)+bensulide, compound of formula (I)+bentazone, compound of formula (I)+benzfendizone, compound of formula (I)+benzobicyclon, compound of formula (I)+benzofenap, compound of formula (I)+bicyclopyrone, compound of formula (I)+bifenox, compound of formula (I)+bilanafos, compound of formula (I)+bispyribac, compound of formula (I)+bispyribac-sodium, compound of formula (I)+borax, compound of formula (I)+bromacil, compound of formula (I)+bromobutide, formula (I)+bromophenoxim, compound of formula (I)+bromoxynil, compound of formula (I)+butachlor, compound of formula (I)+butafenacil, compound of formula (I)+butamifos, compound of formula (I)+butralin, compound of formula (I)+butroxydim, compound of formula (I)+butylate, compound of formula (I)+cacodylic acid, compound of formula (I)+calcium chlorate, compound of formula (I)+cafenstrole, compound of formula (I)+carbetamide, compound of formula (I)+carfentrazone, compound of formula (I)+carfentrazone-ethyl, compound of formula (I)+CDEA, compound of formula (I)+CEPC, compound of formula (I)+chlorflurenol, compound of formula (I)+chlorflurenol-methyl, compound of formula (I)+chloridazon, compound of formula (I)+chlorimuron, compound of formula (I)+chlorimuron-ethyl, compound of formula (I)+chloroacetic acid, compound of formula (I)+chlorotoluron, compound of formula (I)+chlorpropham, compound of formula (I)+chlorsulfuron, compound of formula (I)+chlorthal, compound of formula (I)+chlorthal-dimethyl, compound of formula (I)+cinidon-ethyl, compound of formula (I)+cinmethylin, compound of formula (I)+cinosulfuron, compound of formula (I)+cisanilide, compound of formula (I)+clethodim, compound of formula (I)+clodinafop, compound of formula (I)+clodinafop-propargyl, compound of formula (I)+clomazone, compound of formula (I)+clomeprop, compound of formula (I)+clopyralid, compound of formula (I)+cloransulam, compound of formula (I)+cloransulam-methyl, compound of formula (I)+CMA, compound of formula (I)+4-CPB, compound of formula (I)+CPMF, compound of formula (I)+4-CPP, compound of formula (I)+CPPC, compound of formula (I)+cresol, compound of formula (I)+cumyluron, compound of formula (I)+cyanamide, compound of formula (I)+cyanazine, compound of formula (I)+cycloate, compound of formula (I)+cyclosulfamuron, compound of formula (I)+cycloxydim, compound of formula (I)+cyhalofop, compound of formula (I)+cyhalofop-butyl, compound of formula (I)+2,4-D, compound of formula (I)+3,4-DA, compound of formula (I)+daimuron, compound of formula (I)+dalapon, compound of formula (I)+dazomet, compound of formula (I)+2,4-DB, compound of formula (I)+3,4-DB, compound of formula (I)+2,4-DEB, compound of formula (I)+desmedipham, formula (I)+desmetryn, compound of formula (I)+dicamba, compound of formula (I)+dichlobenil, compound of formula (I)+ortho-dichlorobenzene, compound of formula (I)+para-dichlorobenzene, compound of formula (I)+dichlorprop, compound of formula (I)+dichlorprop-P, compound of formula (I)+diclofop, compound of formula (I)+diclofop-methyl, compound of formula (I)+diclosulam, compound of formula (I)+difenzoquat, compound of formula (I)+difenzoquat metilsulfate, compound of formula (I)+diflufenican, compound of formula (I)+diflufenzopyr, compound of formula (I)+dimefuron, compound of formula (I)+dimepiperate, compound of formula (I)+dimethachlor, compound of formula (I)+dimethametryn, compound of formula (I)+dimethenamid, compound of formula (I)+dimethenamid-P, compound of formula (I)+dimethipin, compound of formula (I)+dimethylarsinic acid, compound of formula (I)+dinitramine, compound of formula (I)+dinoterb, compound of formula (I)+diphenamid, formula (I)+dipropetryn, compound of formula (I)+diquat, compound of formula (I)+diquat dibromide, compound of formula (I)+dithiopyr, compound of formula (I)+diuron, compound of formula (I)+DNOC, compound of formula (I)+3,4-DP, compound of formula (I)+DSMA, compound of formula (I)+EBEP, compound of formula (I)+endothal, compound of formula (I)+EPTC, compound of formula (I)+esprocarb, compound of formula (I)+ethalfluralin, compound of formula (I)+ethametsulfuron, compound of formula (I)+ethametsulfuron-methyl, formula (I)+ethephon, compound of formula (I)+ethofumesate, compound of formula (I)+ethoxyfen, compound of formula (I)+ethoxysulfuron, compound of formula (I)+etobenzanid, compound of formula (I)+fenoxaprop, compound of formula (I)+fenoxaprop-P, compound of formula (I)+fenoxaprop-ethyl, compound of formula (I)+fenoxaprop-P-ethyl, compound of formula (I)+fentrazamide, compound of formula (I)+ferrous sulfate, compound of formula (I)+flamprop-M, compound of formula (I)+flazasulfuron, compound of formula (I)+florasulam, compound of formula (I)+fluazifop, compound of formula (I)+fluazifop-butyl, compound of formula (I)+fluazifop-P, compound of formula (I)+fluazifop-P-butyl, formula (I)+fluazolate, compound of formula (I)+flucarbazone, compound of formula (I)+flucarbazone-sodium, compound of formula (I)+flucetosulfuron, compound of formula (I)+fluchloralin, compound of formula (I)+flufenacet, compound of formula (I)+flufenpyr, compound of formula (I)+flufenpyr-ethyl, formula (I)+flumetralin, compound of formula (I)+flumetsulam, compound of formula (I)+flumiclorac, compound of formula (I)+flumiclorac-pentyl, compound of formula (I)+flumioxazin, formula (I)+flumipropin, compound of formula (I)+fluometuron, compound of formula (I)+fluoroglycofen, compound of formula (I)+fluoroglycofen-ethyl, formula (I)+fluoxaprop, formula (I)+flupoxam, formula (I)+flupropacil, compound of formula (I)+flupropanate, compound of formula (I)+flupyrsulfuron, compound of formula (I)+flupyrsulfuron-methyl-sodium, compound of formula (I)+flurenol, compound of formula (I)+fluridone, compound of formula (I)+flurochloridone, compound of formula (I)+fluroxypyr, compound of formula (I)+flurtamone, compound of formula (I)+fluthiacet, compound of formula (I)+fluthiacet-methyl, compound of formula (I)+fomesafen, compound of formula (I)+foramsulfuron, compound of formula (I)+fosamine, compound of formula (I)+glufosinate, compound of formula (I)+glufosinate-ammonium, compound of formula (I)+glyphosate, compound of formula (I)+haluauxifen, compound of formula (I)+halauxifen-methyl, compound of formula (I)+halosulfuron, compound of formula (I)+halosulfuron-methyl, compound of formula (I)+haloxyfop, compound of formula (I)+haloxyfop-P, compound of formula (I)+HC-252, compound of formula (I)+hexazinone, compound of formula (I)+imazamethabenz, compound of formula (I)+imazamethabenz-methyl, compound of formula (I)+imazamox, compound of formula (I)+imazapic, compound of formula (I)+imazapyr, compound of formula (I)+imazaquin, compound of formula (I)+imazethapyr, compound of formula (I)+imazosulfuron, compound of formula (I)+indanofan, compound of formula (I) and indaziflam, compound of formula (I)+iodomethane, compound of formula (I)+iodosulfuron, compound of formula (I)+iodosulfuron-methyl-sodium, compound of formula (I)+ioxynil, compound of formula (I) and ipfencarbazone, compound of formula (I)+isoproturon, compound of formula (I)+isouron, compound of formula (I)+isoxaben, compound of formula (I)+isoxachlortole, compound of formula (I)+isoxaflutole, formula (I)+isoxapyrifop, compound of formula (I)+karbutilate, compound of formula (I)+lactofen, compound of formula (I)+lenacil, compound of formula (I)+linuron, compound of formula (I)+MAA, compound of formula (I)+MAMA, compound of formula (I)+MCPA, compound of formula (I)+MCPA-thioethyl, compound of formula (I)+MCPB, compound of formula (I)+mecoprop, compound of formula (I)+mecoprop-P, compound of formula (I)+mefenacet, compound of formula (I)+mefluidide, compound of formula (I)+mesosulfuron, compound of formula (I)+mesosulfuron-methyl, compound of formula (I)+mesotrione, compound of formula (I)+metam, compound of formula (I)+metamifop, compound of formula (I)+metamitron, compound of formula (I)+metazachlor, compound of formula (I) and metazosulfuron, compound of formula (I)+methabenzthiazuron, formula (I)+methazole, a compound of formula (I) and methiozolin, compound of formula (I)+methylarsonic acid, compound of formula (I)+methyldymron, compound of formula (I)+methyl isothiocyanate, compound of formula (I)+metobenzuron, formula (I)+metobromuron, compound of formula (I)+metolachlor, compound of formula (I)+S-metolachlor, compound of formula (I)+metosulam, compound of formula (I)+metoxuron, compound of formula (I)+metribuzin, compound of formula (I)+metsulfuron, compound of formula (I)+metsulfuron-methyl, compound of formula (I)+MK-616, compound of formula (I)+molinate, compound of formula (I)+monolinuron, a compound of formula (I) and monosulfuron, a compound of formula (I) and monosulfuron-ester compound of formula (I)+MSMA, compound of formula (I)+naproanilide, compound of formula (I)+napropamide, compound of formula (I)+naptalam, formula (I)+NDA-402989, compound of formula (I)+neburon, compound of formula (I)+nicosulfuron, formula (I)+nipyraclofen, formula (I)+n-methyl glyphosate, compound of formula (I)+nonanoic acid, compound of formula (I)+norflurazon, compound of formula (I)+oleic acid (fatty acids), compound of formula (I)+orbencarb, compound of formula (I)+orthosulfamuron, compound of formula (I)+oryzalin, compound of formula (I)+oxadiargyl, compound of formula (I)+oxadiazon, compound of formula (I)+oxasulfuron, compound of formula (I)+oxaziclomefone, compound of formula (I)+oxyfluorfen, compound of formula (I)+paraquat, compound of formula (I)+paraquat dichloride, compound of formula (I)+pebulate, compound of formula (I)+pendimethalin, compound of formula (I)+penoxsulam, compound of formula (I)+pentachlorophenol, compound of formula (I)+pentanochlor, compound of formula (I)+pentoxazone, compound of formula (I)+pethoxamid, compound of formula (I)+petrolium oils, compound of formula (I)+phenmedipham, compound of formula (I)+phenmedipham-ethyl, compound of formula (I)+picloram, compound of formula (I)+picolinafen, compound of formula (I)+pinoxaden, compound of formula (I)+piperophos, compound of formula (I)+potassium arsenite, compound of formula (I)+potassium azide, compound of formula (I)+pretilachlor, compound of formula (I)+primisulfuron, compound of formula (I)+primisulfuron-methyl, compound of formula (I)+prodiamine, compound of formula (I)+profluazol, compound of formula (I)+profoxydim, formula (I)+prohexadione-calcium, compound of formula (I)+prometon, compound of formula (I)+prometryn, compound of formula (I)+propachlor, compound of formula (I)+propanil, compound of formula (I)+propaquizafop, compound of formula (I)+propazine, compound of formula (I)+propham, compound of formula (I)+propisochlor, compound of formula (I)+propoxycarbazone, compound of formula (I)+propoxycarbazone-sodium, compound of formula (I)+propyzamide, compound of formula (I)+prosulfocarb, compound of formula (I)+prosulfuron, compound of formula (I)+pyraclonil, compound of formula (I)+pyraflufen, compound of formula (I)+pyraflufen-ethyl, formula (I)+pyrasulfotole, compound of formula (I)+pyrazolynate, compound of formula (I)+pyrazosulfuron, compound of formula (I)+pyrazosulfuron-ethyl, compound of formula (I)+pyrazoxyfen, compound of formula (I)+pyribenzoxim, compound of formula (I)+pyributicarb, compound of formula (I)+pyridafol, compound of formula (I)+pyridate, compound of formula (I)+pyriftalid, compound of formula (I)+pyriminobac, compound of formula (I)+pyriminobac-methyl, compound of formula (I)+pyrimisulfan, compound of formula (I)+pyrithiobac, compound of formula (I)+pyrithiobac-sodium, formula (I)+pyroxasulfone, formula (I)+pyroxulam, compound of formula (I)+quinclorac, compound of formula (I)+quinmerac, compound of formula (I)+quinoclamine, compound of formula (I)+quizalofop, compound of formula (I)+quizalofop-P, compound of formula (I)+quizalofop-ethyl, compound of formula (I)+quizalofop-P-ethyl, compound of formula (I)+rimsulfuron, compound of formula (I)+saflufenacil, compound of formula (I)+sethoxydim, compound of formula (I)+siduron, compound of formula (I)+simazine, compound of formula (I)+simetryn, compound of formula (I)+SMA, compound of formula (I)+sodium arsenite, compound of formula (I)+sodium azide, compound of formula (I)+sodium chlorate, compound of formula (I)+sulcotrione, compound of formula (I)+sulfentrazone, compound of formula (I)+sulfometuron, compound of formula (I)+sulfometuron-methyl, compound of formula (I)+sulfosate, compound of formula (I)+sulfosulfuron, compound of formula (I)+sulfuric acid, compound of formula (I)+tar oils, compound of formula (I)+2,3,6-TBA, compound of formula (I)+TCA, compound of formula (I)+TCA-sodium, formula (I)+tebutam, compound of formula (I)+tebuthiuron, formula (I)+tefuryltrione, compound of formula 1+tembotrione, compound of formula (I)+tepraloxydim, compound of formula (I)+terbacil, compound of formula (I)+terbumeton, compound of formula (I)+terbuthylazine, compound of formula (I)+terbutryn, compound of formula (I)+thenylchlor, compound of formula (I)+thiazafluron, compound of formula (I)+thiazopyr, compound of formula (I)+thifensulfuron, compound of formula (I)+thiencarbazone, compound of formula (I)+thifensulfuron-methyl, compound of formula (I)+thiobencarb, compound of formula (I)+tiocarbazil, compound of formula (I)+topramezone, compound of formula (I)+tralkoxydim, a compound of formula (I) and triafamone compound of formula (I)+tri-allate, compound of formula (I)+triasulfuron, compound of formula (I)+triaziflam, compound of formula (I)+tribenuron, compound of formula (I)+tribenuron-methyl, compound of formula (I)+tricamba, compound of formula (I)+triclopyr, compound of formula (I)+trietazine, compound of formula (I)+trifloxysulfuron, compound of formula (I)+trifloxysulfuron-sodium, compound of formula (I)+trifluralin, compound of formula (I)+triflusulfuron, compound of formula (I)+triflusulfuron-methyl, compound of formula (I)+trifop, compound of formula (I)+trifop-methyl, compound of formula (I)+trihydroxytriazine, compound of formula (I)+trinexapac-ethyl, compound of formula (I)+tritosulfuron, compound of formula (I)+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (CAS RN 353292-31-6) and the compound of formula (I)+VX-573.

In particular, the following mixtures are important:

mixtures of a compound of formula (I) with an acetanilide (e.g. compound of formula (I)+acetochlor, compound of formula (I)+dimethenamid, compound of formula (I)+metolachlor, compound of formula (I)+S-metolachlor, or compound of formula (I)+pretilachlor) or with other inhibitors of VLCFAE (e.g. compound of formula (I)+pyroxasulfone);

mixtures of a compound of formula (I) with an HPPD inhibitor (e.g. compound of formula (I)+isoxaflutole, compound of formula (I)+mesotrione, compound of formula (I)+pyrasulfotole, compound of formula (I)+sulcotrione, compound of formula (I)+tembotrione, compound of formula (I)+topramezone, compound of formula (I)+bicyclopyrone;

mixtures of a compound of formula (I) with a PSII inhibitor (e.g. compound of formula (I)+atrazine, compound of formula (I)+terbuthylazine, compound of formula (I)+ametrin, compound of formula (I)+bromoxinyl);

mixtures of a compound of formula (I) with glyphosate;

mixtures of a compound of formula (I) with glufosinate-ammonium;

mixtures of a compound of formula (I) with a PPO inhibitor (e.g. compound of formula (I)+acifluorfen-sodium, compound of formula (I)+butafenacil, compound of formula (I)+carfentrazone-ethyl, compound of formula (I)+cinidon-ethyl, compound of formula (I)+flumioxazin, compound of formula (I)+fomesafen, compound of formula (I)+lactofen, or compound of formula (I)+SYN 523 ([3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester) (CAS RN 353292-31-6)).

Whilst two-way mixtures of a compound of formula (I) and another herbicide are explicitly disclosed above, the skilled man will appreciate that the invention extends to three-way, and further multiple combinations comprising the above two-way mixtures. In particular, the invention extends to:

mixtures of a compound of formula (I) with a PSII inhibitor and an HPPD inhibitor (e.g. compound of formula (I)+PSII inhibitor+isoxaflutole, compound of formula (I)+PSII inhibitor+mesotrione, compound of formula (I)+PSII inhibitor+pyrasulfotole, compound of formula (I)+PSII inhibito+sulcotrione, compound of formula (I)+PSII inhibitor+tembotrione, compound of formula (I)+PSII inhibitor+topramezone, compound of formula (I)+PSII inhibitor+bicyclopyrone;

mixtures of a compound of formula (I) with glyphosate and an HPPD inhibitor (e.g. compound of formula (I)+glyphosate+isoxaflutole, compound of formula (I)+glyphosate+mesotrione, compound of formula (I)+glyphosate+pyrasulfotole, compound of formula (I)+glyphosate+sulcotrione, compound of formula (I)+glyphosate+tembotrione, compound of formula (I)+glyphosate+topramezone, compound of formula (I)+glyphosate+bicyclopyrone

mixtures of a compound of formula (I) with glufosinate-ammonium and an HPPD inhibitor (e.g. compound of formula (I)+glufosinate-ammonium+isoxaflutole, compound of formula (I)+glufosinate-ammonium+mesotrione, compound of formula (I)+glufosinate-ammonium+pyrasulfotole, compound of formula (I)+glufosinate-ammonium+sulcotrione, compound of formula (I)+glufosinate-ammonium+tembotrione, compound of formula (I)+glufosinate-ammonium+topramezone, compound of formula (I)+glufosinate-ammonium+bicyclopyrone;

mixtures of a compound of formula (I) with a VLCFAE inhibitor and an HPPD inhibitor (e.g. compound of formula (I)+S-metolachlor+isoxaflutole, compound of formula (I)+S-metolachlor+mesotrione, compound of formula (I)+S-metolachlor+pyrasulfotole, compound of formula (I)+S-metolachlor+sulcotrione, compound of formula (I)+S-metolachlor+tembotrione, compound of formula (I)+S-metolachlor+topramezone, compound of formula (I)+S-metolachlor+bicyclopyrone, compound of formula (I)+acetochlor+isoxaflutole, compound of formula (I)+acetochlor+mesotrione, compound of formula (I)+acetochlor+pyrasulfotole, compound of formula (I)+acetochlor+sulcotrione, compound of formula (I)+acetochlor+tembotrione, compound of formula (I)+acetochlor+topramezone, compound of formula (I)+acetochlor+bicyclopyrone, compound of formula (I)+pyroxasulfone+isoxaflutole, compound of formula (I)+pyroxasulfone+mesotrione, compound of formula (I)+pyroxasulfone+pyrasulfotole, compound of formula (I)+pyroxasulfone+sulcotrione, compound of formula (I)+pyroxasulfone+tembotrione, compound of formula (I)+pyroxasulfone+topramezone, compound of formula (I)+pyroxasulfone+bicyclopyrone,

Particularly preferred are mixtures of the compound of formula (I) with mesotrione, bicyclopyrone, isoxaflutole, tembotrione, topramezone, sulcotrione, pyrasulfotole, metolachlor, S-metolachlor, acetochlor, pretilachlor, pyroxasulfone, P-dimethenamid, dimethenamid, flufenacet, pethoxamid, atrazine, terbuthylazine, bromoxynil, metribuzin, amicarbazone, bentazone, ametryn, hexazinone, diuron, tebuthiuron, glyphosate, paraquat, diquat, glufosinate, acifluorfen-sodium, butafenacil, carfentrazone-ethyl, cinidon-ethyl, flumioxazin, fomesafen, lactofen, [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester.

The mixing partners of the compound of formula (I) may also be in the form of esters or salts, as mentioned e.g. in The Pesticide Manual, 14th Edition (BCPC), 2006. The reference to acifluorfen-sodium also applies to acifluorfen, the reference to dimethenamid also applies to dimethenamid-P, the reference to glufosinate-ammonium also applies to glufosinate, the reference to bensulfuron-methyl also applies to bensulfuron, the reference to cloransulam-methyl also applies to cloransulam, the reference to flamprop-M also applies to flamprop, and the reference to pyrithiobac-sodium also applies to pyrithiobac, etc.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

The compounds of formula (I) according to the invention can also be used in combination with one or more safeners. Likewise, mixtures of a compound of formula (I) according to the invention with one or more further active ingredients, in particular with one or more further herbicides, can also be used in combination with one or more safeners. The term “safener” as used herein means a chemical that when used in combination with a herbicide reduces the undesirable effects of the herbicide on non-target organisms, for example, a safener protects crops from injury by herbicides but does not prevent the herbicide from killing the weeds. Where a compound of formula (I) is combined with a safener, the following combinations of the compound of formula (I) and the safener are particularly preferred. Compound of formula (I)+AD 67 (MON 4660), compound of formula (I)+benoxacor, compound of formula (I)+cloquintocet-mexyl, compound of formula (I)+cyometrinil and a compound of formula (I)+the corresponding (Z) isomer of cyometrinil, compound of formula (I)+cyprosulfamide (CAS RN 221667-31-8), compound of formula (I)+dichlormid, compound of formula (I) and dicyclonon, compound of formula (I) and dietholate, compound of formula (I)+fenchlorazole-ethyl, compound of formula (I)+fenclorim, compound of formula (I)+flurazole, compound of formula (I)+fluxofenim, compound of formula (I)+furilazole and a compound of formula (I)+the corresponding R isomer or furilazome, compound of formula (I)+isoxadifen-ethyl, compound of formula (I)+mefenpyr-diethyl, compound of formula (I) and mephenate, compound of formula (I)+oxabetrinil, compound of formula (I)+naphthalic anhydride (CAS RN 81-84-5), compound of formula (I) and TI-35, compound of formula (I)+N-isopropyl-4-(2-methoxy-benzoylsulfamoyl)-benzamide (CAS RN 221668-34-4) and a compound of formula (I)+N-(2-methoxybenzoyl)-4-[(methylaminocarbonyl)amino]benzenesulfonamide. Particularly preferred are mixtures of a compound of formula (I) with benoxacor, a compound of formula (I) with cloquintocet-mexyl, a compound of formula (I)+cyprosulfamide and a compound of formula (I) with N-(2-methoxybenzoyl)-4-[(methylaminocarbonyl)amino]benzenesulfonamide.

The safeners of the compound of formula (I) may also be in the form of esters or salts, as mentioned e.g. in The Pesticide Manual, 14th Edition (BCPC), 2006. The reference to cloquintocet-mexyl also applies to cloquintocet and to a lithium, sodium, potassium, calcium, magnesium, aluminium, iron, ammonium, quaternary ammonium, sulfonium or phosphonium salt thereof as disclosed in WO02/34048 and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) and any further active ingredient, in particular a further herbicide, with the safener).

It is possible that the safener and a compound of formula (I) and one or more additional herbicide(s), if any, are applied simultaneously. For example, the safener, a compound of formula (I) and one or more additional herbicide(s), if any, might be applied to the locus pre-emergence or might be applied to the crop post-emergence. It is also possible that the safener and a compound of formula (I) and one or more additional herbicide(s), if any, are applied sequentially. For example, the safener might be applied before sowing the seeds as a seed treatment and a compound of formula (I) and one or more additional herbicides, if any, might be applied to the locus pre-emergence or might be applied to the crop post-emergence.

Preferred mixtures of a compound of formula (I) with further herbicides and safeners include:

Mixtures of a compound of formula (I) with S-metolachlor and a safener, particularly benoxacor.

Mixtures of a compound of formula (I) with isoxaflutole and a safener.

Mixtures of a compound of formula (I) with mesotrione and a safener.

Mixtures of a compound of formula (I) with sulcotrione and a safener.

Mixtures of a compound of formula (I) with tembotrione and a safener.

Mixtures of a compound of formula (I) with topramezone and a safener.

Mixtures of a compound of formula (I) with bicyclopyrone and a safener.

Mixtures of a compound of formula (I) with a PSII inhibitor and a safener.

Mixtures of a compound of formula (I) with a PSII inhibitor and isoxaflutole and a safener.

Mixtures of a compound of formula (I) with a PSII inhibitor and mesotrione and a safener.

Mixtures of a compound of formula (I) with a PSII inhibitor and sulcotrione and a safener.

Mixtures of a compound of formula (I) with a PSII inhibitor and tembotrione and a safener.

Mixtures of a compound of formula (I) with a PSII inhibitor and topramezone and a safener.

Mixtures of a compound of formula (I) with a PSII inhibitor and bicyclopyrone and a safener.

Mixtures of a compound of formula (I) with glyphosate and a safener.

Mixtures of a compound of formula (I) with glyphosate and isoxaflutole and a safener.

Mixtures of a compound of formula (I) with glyphosate and mesotrione and a safener.

Mixtures of a compound of formula (I) with glyphosate and sulcotrione and a safener.

Mixtures of a compound of formula (I) with glyphosate and tembotrione and a safener.

Mixtures of a compound of formula (I) with glyphosate and topramezone and a safener.

Mixtures of a compound of formula (I) with glyphosate and bicyclopyrone and a safener.

Mixtures of a compound of formula (I) with glufosinate-ammonium and a safener.

Mixtures of a compound of formula (I) with glufosinate-ammonium and isoxaflutole and a safener.

Mixtures of a compound of formula (I) with glufosinate-ammonium and mesotrione and a safener.

Mixtures of a compound of formula (I) with glufosinate-ammonium and sulcotrione and a safener.

Mixtures of a compound of formula (I) with glufosinate-ammonium and tembotrione and a safener.

Mixtures of a compound of formula (I) with glufosinate-ammonium and topramezone and a safener.

Mixtures of a compound of formula (I) with glufosinate-ammonium and bicyclopyrone and a safener.

Mixtures of a compound of formula (I) with S-metolachlor and a safener.

Mixtures of a compound of formula (I) with S-metolachlor and isoxaflutole and a safener.

Mixtures of a compound of formula (I) with S-metolachlor and mesotrione and a safener.

Mixtures of a compound of formula (I) with S-metolachlor and sulcotrione and a safener.

Mixtures of a compound of formula (I) with S-metolachlor and tembotrione and a safener.

Mixtures of a compound of formula (I) with S-metolachlor and topramezone and a safener.

Mixtures of a compound of formula (I) with S-metolachlor and bicyclopyrone and a safener

Mixtures of a compound of formula (I) with pyroxasulfone and a safener.

Mixtures of a compound of formula (I) with pyroxasulfone and isoxaflutole and a safener.

Mixtures of a compound of formula (I) with pyroxasulfone and mesotrione and a safener.

Mixtures of a compound of formula (I) with pyroxasulfone and sulcotrione and a safener.

Mixtures of a compound of formula (I) with pyroxasulfone and tembotrione and a safener.

Mixtures of a compound of formula (I) with pyroxasulfone and topramezone and a safener.

Mixtures of a compound of formula (I) with pyroxasulfone and bicyclopyrone and a safener

Mixtures of a compound of formula (I) with acetochlor and a safener.

Mixtures of a compound of formula (I) with acetochlor and isoxaflutole and a safener.

Mixtures of a compound of formula (I) with acetochlor and mesotrione and a safener.

Mixtures of a compound of formula (I) with acetochlor and sulcotrione and a safener.

Mixtures of a compound of formula (I) with acetochlor and tembotrione and a safener.

Mixtures of a compound of formula (I) with acetochlor and topramezone and a safener.

Mixtures of a compound of formula (I) with acetochlor and bicyclopyrone and a safener.

Various aspects and embodiments of the present invention will now be illustrated in more detail by way of example. It will be appreciated that modification of detail may be made without departing from the scope of the invention.

For the avoidance of doubt, where a literary reference, patent application, or patent, is cited within the text of this application, the entire text of said citation is herein incorporated by reference.

### EXAMPLES

### Preparation Examples

The following abbreviations were used in this section: s=singlet; bs=broad singlet; d=doublet; dd=double doublet; dt=double triplet; t=triplet, tt=triple triplet, q=quartet, sept=septet; m=multiplet; RT=retention time, MH⁺=molecular mass of the molecular cation.

1H NMR spectra were recorded at 400 MHz on a Varian Unity Inova instrument.

### Example 1

### Preparation of 1-(5-tert-butyl-1-methyl-1H-pyrazol-3-yl)-4-chloro-5-hydroxy-3-methyl-1,5-dihydro-pyrrol-2-one (B2)

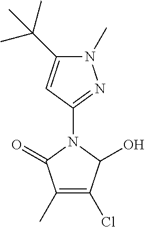 [SMILES: CC1=C(Cl)C(O)N(c2cc(C(C)(C)C)n(C)n2)C1=O]

### Procedure for synthesis of 4-Hydroxy-3-methyl-5H-furan-2-one (Step-1)

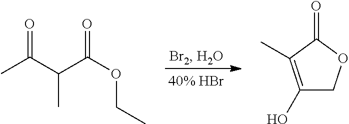

To a three neck 100 mL round bottom flask, ethyl 2-methyl-3-oxo-butanoate (20 g, 138.72 mmol) was suspended in water (40 mL) and cooled to 0° C. Bromine (7.100 mL, 138.70 mmol) was added to the reaction mixture slowly. The reaction mixture was stirred at room temperature for overnight. After the completion of the reaction, tertiary-butyl methyl ether (100 mL) was added and organic layer was separated which was washed with aqueous sodium thiosulfate (1 M, 50 mL), dried over sodium sulfate, concentrated on rotovap to give colorless liquid (27 g) to which (0.5 mL) was added and the reaction mass was refluxed for overnight. The solid formed was filtered and washed with tertiary-butyl methyl ether (10 mL×3) to give the desired product (8.0 g, 51%) as a white solid.

¹H NMR (DMSO-D₆, 400 MHz): δ 11.78 (s, 1H), δ 4.56 (dd, 2H), δ 1.57 (t, 3H).

### Procedure for synthesis of 4-Chloro-3-methyl-5H-furan-2-one (Step-2)

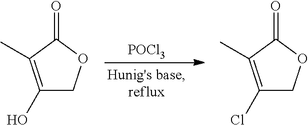

To a two neck 50 mL round bottom flask, 3-hydroxy-4-methyl-2H-furan-5-one (3 g, 26.31 mmol) was dissolved in phosphorus oxychloride (15 mL, 158.85 mmol) and then N,N-diisopropyl ethylamine (4.58 mL, 26.31 mmol) was added slowly to it and the reaction mixture was refluxed for 6 h. Phosphorus oxychloride was evaporated under vacuum and the material was extracted with tertiary-butyl methyl ether (50 mL×3). The organic layer was dried over sodium sulfate, concentrated under reduced pressure to furnish crude product (2.4 g) which was purified by flash chromatography using ethyl acetate/hexane (0-60%) as mobile phase yielding pure compound (2.3 g, 66%) as colorless crystal.

¹H NMR (CDCl₃, 400 MHz): δ 4.73 (dd, 2H), δ 1.91 (t, 2H).

GCMS: m/z 131.9 (M).

### Procedure for synthesis of 5-Bromo-4-chloro-3-methyl-5H-furan-2-one (Step-3)

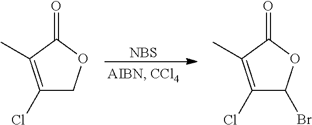

To a 50 mL three neck round bottom flask, 3-chloro-4-methyl-2H-furan-5-one (0.396 g, 3 mmol) was dissolved in carbon tetrachloride (20 mL). NBS (0.531 g, 3 mmol) and Azoisobutyronitrile (0.072 g, 14 mol %) were added to it and reaction mixture was refluxed for 3 days. Reaction mass was filtered and concentrated and purified by flash chromatography using ethyl acetate: hexane (0-30%) as mobile phase yielding compound (0.385 g, 60%) as colorless liquid.

¹H NMR (CDCl₃, 400 MHz): δ 6.70 (dd, 1H), δ 1.98 (d, 3H).

### Procedure for synthesis of (Z)-3-Chloro-4,4-dimethyl-pent-2-enenitrile (Step-4)

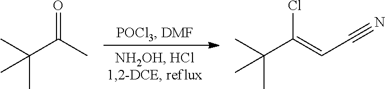

To a three-neck round bottomed flask, equipped with a nitrogen inlet was added dimethylformamide (80 mL, 1000 mmol). The reaction mixture was cooled to 0° C. Phosphorus oxychloride (2.4 equiv, 958.47 mmol) was added dropwise at such a rate to keep temperature below 25° C. with constant stirring with mechanical stirrer. A solution of 3,3-dimethylbutan-2-one (40 g, 399.36 mmol) in 1,2-dichloroethane (140 mL, 1750 mmol) was added dropwise and reaction mass was heated at 50-55° C. for 2 h and then at 70-75° C. for another 2 h. Hydroxylammonium chloride (2.4 equiv., 958.5 mmol) was added to to reaction mass portion wise. Care should be taken for exotherm (temperature rises up to 80° C.). Reaction mixture was then heated to reflux 80° C. for 2 h then cooled down. Water (300 mL) was added to the reaction followed by tertiary-butyl methyl ether (250 mL). Some sticky material was formed initially which got dissolved after stirring for 3-4 hr. The organic layer was separated and water layer was again extracted with tertiary-butyl methyl ether (250 mL×2). Combined organic layers were dried over anhydrous sodium sulfate (50 g) and concentrated to give crude material (54 g, 94%). Distillation at 90-95° C. at 89 mbar pressure furnished pure product (32 g, 56%) as colorless liquid.

¹H NMR (CDCl₃, 400 MHz): δ 5.55 (s, 1H), δ 1.22 (s, 9H). GCMS: m/z 142.9 (M)

### Procedure for synthesis of 5-tert-butyl-1-methyl-1H-pyrazol-3-ylamine (Step-5)

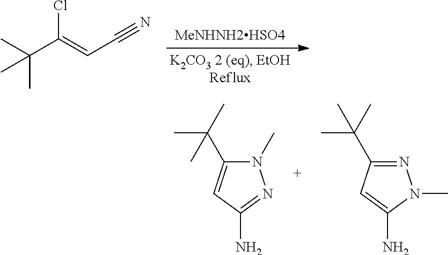

To a two neck 50 mL round bottom flask, methylhydrazine sulfate (1.4 g, 10 mmol) was suspended in ethanol (5 mL) to which potassium carbonate (2.76 g, 20 mmol) was added and reaction mass was stirred at room temperature for 1 h. Solution of (Z)-3-chloro-4,4-dimethyl-pent-2-enenitrile (1.4 g, 10 mmol) in ethanol (5 mL) was added drop wise to the above reaction mass and was heated to reflux for additional 3 hr. Reaction mass was filtered to remove potassium carbonate and washed with methanol. Filtrate was concentrated and purified by flash chromatography using hexane ethyl acetate (0-100%) giving pure 5-tert-butyl-1-methyl-pyrazol-3-amine (0.830 g, 54%)¹H NMR (CDCl₃, 400 MHz) δ 5.38 (s, 1H), δ 3.75 (s, 3H), δ 1.31 (s, 9H) and 5-tert-butyl-2-methyl-pyrazol-3-amine (0.200 g, 13%). ¹H NMR (CDCl₃, 400 MHz) δ 5.42 (s, 1H), δ 3.63 (s, 3H), δ 1.25 (s, 9H).

LCMS for compound I (ESI, rt=0.68 min): m/z 154.0 (M+1). LCMS for compound II (ESI, rt=0.51 min): m/z 154.0 (M+1).

### Procedure for synthesis of 5-(5-tert-butyl-1-methyl-1H-pyrazol-3-yl-amino)-4-chloro-3-methyl-5H-furan-2-one (Step-6)

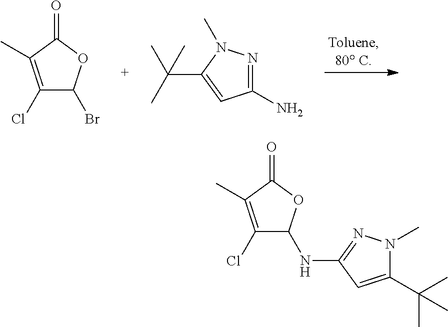

To a three neck 50 mL round bottom flask, 2-bromo-3-chloro-4-methyl-2H-furan-5-one (2 g, 9.4589 mmol) was dissolved in toluene (19 mL) to which a solution of 5-tert-butyl-1-methyl-pyrazol-3-amine (1.45 g, 9.46 mmol) in toluene (19 mL) was added under nitrogen atmosphere followed by slow addition of Triethylamine (1.3 mL, 9.3 mmol) and the reaction was heated for 2 h at 80° C. and was monitored by TLC for completion. After 2 hr, reaction mass was cooled and filtered to remove the solid and the residue was washed with toluene (5 ml x 4). The filtrate was then concentrated to give the crude material (3 g). The crude material was purified by column chromatography using ethyl acetate in cyclohexane as the eluent to furnish the desired product (1.4 g, 52%) as white gummy solid. ¹H NMR (CDCl₃, 400 MHz): δ 6.17 (dd, 1H), δ 5.53 (s, 1H), δ 4.54 (d, 1H), δ 3.80 (s, 3H), δ 1.95 (d, 3H), δ 1.32 (s, 9H). LCMS (ESI): m/z 284.1 (M+1)

### Procedure for synthesis of Acetic acid-1-(5-tert-butyl)-1-methyl-1H-pyrazol-3-yl)-3-chloro-4-methyl-5-oxo-2,5-dihydro-1H-pyrrol-2-yl-ester (Step-7)

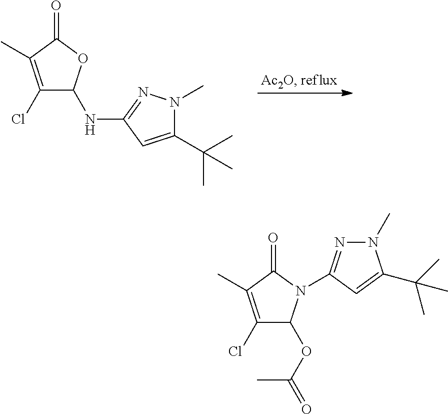 [SMILES: CC1=C(Cl)C(Nc2cc(C(C)(C)C)n(C)n2)OC1=O ; CC(=O)OC1C(Cl)=C(C)C(=O)N1c1cc(C(C)(C)C)n(C)n1]

To a 10 mL round bottom flask, furanone (0.130 g, 0.46 mmol) was dissolved in acetic anhydride (1 mL). The reaction mixture was heated to reflux for 30 min and then quenched with methanol/water (1/3 mL, v/v) and extracted with ethyl acetate (3×20 mL). The organic layer was dried, concentrated under reduced pressure to give crude product (0.140 g) which was purified by flash chromatography using hexane ethyl acetate (0-60%) as mobile phase to furnish pure compound (0.094 g, 63%) as gummy solid. ¹H NMR (CDCl₃, 400 MHz): δ 7.09 (d, 1H), δ 6.41 (s, 1H), δ 3.83 (s, 3H), δ 2.16 (s, 3H), δ 1.95 (d, 3H), δ 1.36 (s, 9H). LCMS (ESI): m/z 326.1 (M+1).

### Procedure for synthesis of 1-(5-tert-butyl-1-methyl-1H-pyrazol-3-yl)-4-chloro-5-hydroxy-3-methyl-1,5-dihydro-pyrrol-2-one (Step-8)

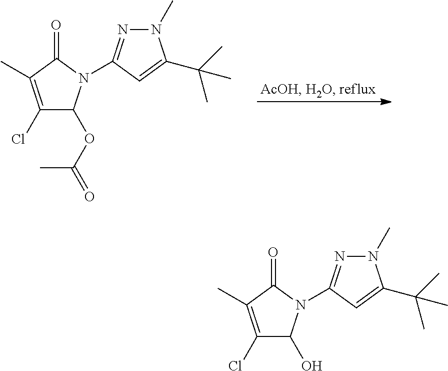 [SMILES: CC(=O)OC1C(Cl)=C(C)C(=O)N1c1cc(C(C)(C)C)n(C)n1 ; CC1=C(Cl)C(O)N(c2cc(C(C)(C)C)n(C)n2)C1=O]

To a 50 mL two neck round bottom flask, [1-(5-tert-butyl-1-methyl-pyrazol-3-yl)-3-chloro-4-methyl-5-oxo-2H-pyrrol-2-yl] acetate (0.574 g, 1.762 mmol) was dissolved in acetic acid (10 mL, 174 mmol) and water 10 mL was added. Reaction mass was heated to reflux for 2 h. Acetic acid and water were evaporated to furnish solid material. To which 35 mL of cyclohexane was added and heated to 80° C., solid material get dissolves then it was filtered. The hot cyclohexane was allow to cool to rt which gave solid precipitate which was filtered to furnish pure product 1-(5-tert-butyl-1-methyl-pyrazol-3-yl)-3-chloro-2-hydroxy-4-methyl-2H-pyrrol-5-one K (0.390 g, 78.0%).

¹H NMR (CDCl₃, 400 MHz): δ 6.49 (s, 1H), δ 5.86 (s, 1H), δ 5.05 (bs, 1H), δ 3.87 (s, 3H), δ 1.94 (d, 3H), δ 1.39 (s, 9H). LCMS: m/z 284.0 (M+1).

### Example 2

### Preparation of 3-chloro-1-[5-(1,1-dimethylbut-3-enyl)-1-methyl-pyrazol-3-yl]-2-hydroxy-4-methyl-2H-pyrrol-5-one (B6)

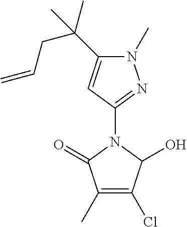 [SMILES: C=CCC(C)(C)c1cc(N2C(=O)C(C)=C(Cl)C2O)nn1C]

### Preparation of (2Z)-2-chloro-4,4-dimethyl-hepta-2,6-dienenitrile and (2E)-2-chloro-4,4-dimethyl-hepta-2,6-dienenitrile (Step 1)

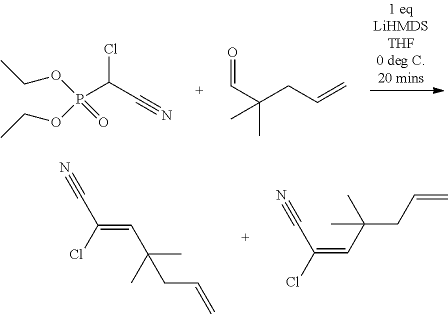

2-chloro-2-diethoxyphosphoryl-acetonitrile (3.80 g, 18.0 mM) was dissolved in 15 ml dry THF then cooled to −10° C. with stirring. Lithium bis(trimethylsilyl)amide (1M in THF, 18.0 ml, 18.0 mM) was added dropwise over 15 minutes to give an amber solution then 2,2-dimethylpent-4-enal (2.12 g, 18.9 mM) was added over 5 minutes and stirred at 0° C. After 20 minutes at 0° C. the reaction was diluted with isohexane (200 ml) then washed sequentially with 2N HCl (aq, 20 ml), water (20 ml), saturated NaHCO3 (aq, 20 ml), water (10 ml), saturated brine (aq, 10 ml), then passed through phase separation cartridge to remove any droplets of water and evaporated to give an amber oil (2.58 g, 84%). ¹H NMR (CDCl₃) showed a 73:27 mixture of geometric isomers:—

Major isomer 6.53 (s, 1H), 5.72 (m, 1H), 5.13 (m, 1H), 5.09 (m, 1H), 2.22 (dm, 2H), 1.28 (s, 6H)

Minor isomer 6.56 (s, 1H), 5.72 (m, 1H), 5.17 (m, 1H), 5.14 (m, 1H), 2.27 (dm, 2H), 1.25 (s, 6H)

### Preparation of 4,4-dimethylhept-6-en-2-ynenitrile (Step 2)

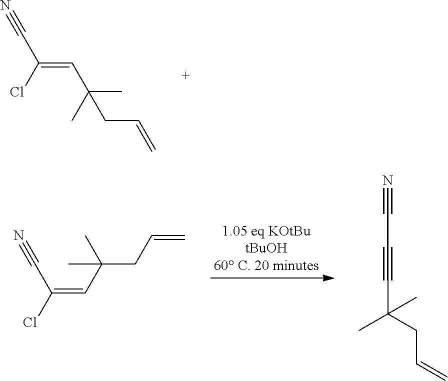

Potassium tert butoxide (1 M in tert butanol, 6.0 ml, 5.97 mM) was added all at once to a mixture of 2-chloro-4,4-dimethyl-hepta-2,6-dienenitrile (73:27 mix of 2-E and 2-Z isomers, 0.965 g, 5.69 mM) and was heated at 60° C. with stirring. After 20 minutes gc showed 4,4-dimethylhept-6-en-2-ynenitrile had formed and was reacted further directly in step 3.

### Preparation of 5-(1,1-dimethylbut-3-enyl)-1-methyl-pyrazol-3-amine (Step 3)

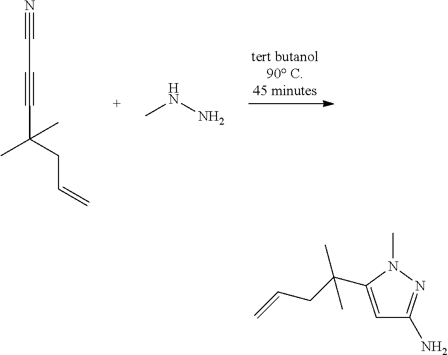

Methyl hydrazine (0.288 g, 6.26 mM) was added to the reaction mixture from step 2 and the reaction was heated at 88° C. with stirring with a reflux condenser fitted. After 45 minutes, the reaction was evaporated and partitioned between water (5 ml), saturated brine (3 ml) and ethyl acetate (20 ml), shaken, then the layers were separated and the aqueous layer was extracted with more ethyl acetate (2×15 ml). The combined ethyl acetate extracts were dried with Na₂SO₄, filtered and the filtrate was evaporated to give a brown gum (1.045 g) which was chromatographed to give an amber gum (0.175 g, 17%). ¹H NMR (CDCl₃) 5.60 (m, 1H), 5.40 (s, 1H), 5.04 (dm, 1H), 5.01 (m, 1H), 3.77 (s, 3H), 3.48 (br s, 2H), 2.39 (dm, 2H), 1.30 (s, 6H)

### Preparation of 3-chloro-1-[5-(1,1-dimethylbut-3-enyl)-1-methyl-pyrazol-3-yl]-4-methyl-pyrrole-2,5-dione (Step 4)

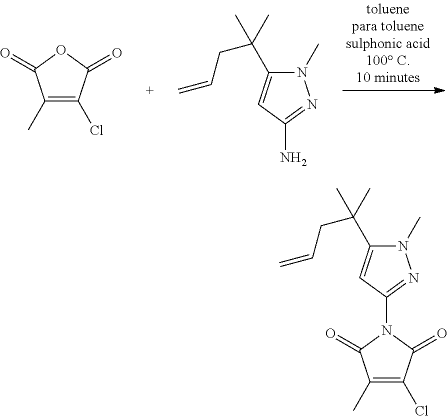

3-chloro-4-methyl-furan-2,5-dione (0.233 g, 1.589 mM) and 5-(1,1-dimethylbut-3-enyl)-1-methyl-pyrazol-3-amine (0.259 g, 1.445 mM) and para toluene sulphonic acid monohydrate (0.003 g, 0.014 mM) were dissolved in toluene (1 ml) and heated with stirring in a microwave at 100° C. for 10 minutes. Ethyl acetate (5 ml) and saturated sodium hydrogen carbonate (aqueous, 1 ml) were added and shaken. The layers were separated and the aqueous layer was extracted with more ethyl acetate (2×2 ml), The combined ethyl acetate extracts were dried with Na₂SO₄, filtered and the filtrate was evaporated to give a pale yellow solid (0.470 g) which was chromatographed to give a pale yellow solid (0.356 g, 80%)¹H NMR (CDCl₃) 6.07 (s, 1H), 5.61 (m, 1H), 5.07 (dm, 1H), 5.04 (m, 1H), 3.98 (s, 3H), 2.45 (dm, 2H), 2.14 (s, 3H), 1.37 (s, 6H).

### Preparation of 3-chloro-1-[5-(1,1-dimethylbut-3-enyl)-1-methyl-pyrazol-3-yl]-2-hydroxy-4-methyl-2H-pyrrol-5-one (B6) and 4-chloro-1-[5-(1,1-dimethylbut-3-enyl)-1-methyl-pyrazol-3-yl]-2-hydroxy-3-methyl-2H-pyrrol-5-one (Step 5)

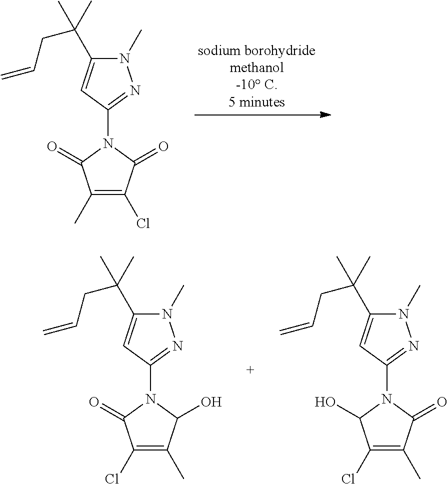

3-chloro-1-[5-(1,1-dimethylbut-3-enyl)-1-methyl-pyrazol-3-yl]-4-methyl-pyrrole-2,5-dione (0.317 g, 1.030 mM) was dissolved in methanol (6 ml) and was cooled to −10° C. with stirring. Sodium borohydride (0.029 g, 0.775 mM) was added. After 5 minutes, acetone was added (0.5 ml) to quench any remaining sodium borohydride. The reaction was evaporated to a give a gum which was partitioned between ethyl acetate (20 ml), water (4 ml) and saturated brine (4 ml) and shaken. The layers were separated and the aqueous layer was extracted with more ethyl acetate (2×10 ml). The combined ethyl acetate extracts were dried with Na₂SO₄, filtered and the filtrate was evaporated to give a pale amber gum (0.410 g) which was chromatographed to give an amber gum, 3-chloro-1-[5-(1,1-dimethylbut-3-enyl)-1-methyl-pyrazol-3-yl]-2-hydroxy-4-methyl-2H-pyrrol-5-one (B6) (0.092 g, 29%)¹H NMR (CDCl₃) 6.49 (s, 1H), 5.87 (m, 1H), 5.59 (m, 1H), 5.07 (m, 1H), 5.03 (m, 1H), 4.97 (d, 1H), 3.87 (s, 3H), 2.44 (d, 2H), 1.95 (s, 3H), 1.37 (s, 6H) and 4-chloro-1-[5-(1,1-dimethylbut-3-enyl)-1-methyl-pyrazol-3-yl]-2-hydroxy-3-methyl-2H-pyrrol-5-one ¹H NMR (CDCl₃) 6.52 (s, 1H), 5.84 (m, 1H), 5.59 (m, 1H), 5.07 (m, 1H), 5.03 (m, 1H), 4.95 (d, 1H), 3.88 (s, 3H), 2.44 (d, 2H), 2.14 (s, 3H), 1.37 (s, 6H).

### Example 3

### Preparation of 4,4-dimethyl-6,7-dihydro-5H-pyrazolo[1,5-a]pyridin-2-amine

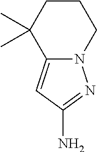

### Preparation of 3,3-dimethyltetrahydropyran-2-one (Step 1)

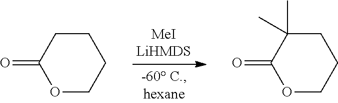

To a solution of δ-valerolactone (0.9279 mL, 10 mmol) and iodomethane (2.49 mL, 40 mmol) in THF (20 mL) at −78° C., was slowly added a solution of LiHMDS (1.0 mol/L) in HEXANE (22 mL, 22 mmol). The addition was complete in 30 minutes keeping the temperature around −60° C. all the time. The reaction was left to slowly warm up and was stirred at rt overnight. Acetic acid (˜2 ml) was added to the reaction (causing the precipitation of white solid) and the whole mixture was concentrated. The residue was purified by column chromatography (gradient of EtOAc in hexane) (860 mg). ¹H NMR (CDCl₃) 4.35 (t, 2H); 1.91 (quint, 2H); 1.76 (t, 2H); 1.30 (s, 6H).

### Preparation of 7-hydroxy-4,4-dimethyl-3-oxo-heptanenitrile (Step 2)

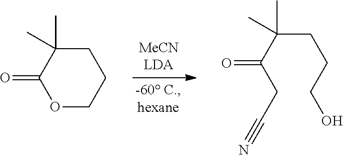

To a solution of N-isopropylpropan-2-amine (1.04 mL, 7.38 mmol) in tetrahydrofuran (13.4 mL) at −78° C., was slowly added a solution of n-butyllithium (3.4 mL, 8.39 mmol). This mixture was stirred for 5 min before adding acetonitrile (0.386 mL, 7.38 mmol) and 10 min later a solution 3,3-dimethyltetrahydropyran-2-one (0.860 g, 6.71 mmol) in THF (6.71 mL). The reaction was then left to slowly warm up and was quenched with NH4Cl after 6 h (−5° C.). The reaction was left sitting at rt overnight. The aqueous layer was then extracted with EtOAc. The combined organic layers were washed with brine, dried over MgSO4 and concentrated. (578 mg)¹H NMR (CDCl₃) 3.92 (dt, 1H); 3.70 (dd, 1H); 2.73 (d, 1H); 2.67 (d, 1H); 2.42 (br s, 1H); 1.92 (dt, 1H); 1.88-1.77 (m, 1H); 1.42-1.37 (m, 1H); 1.29-1.23 (m, 1H); 1.05 (s, 3H); 1.00 (s, 3H).

### Preparation of 3-(5-amino-1H-pyrazol-3-yl)-3-methyl-butan-1-ol (Step 3)

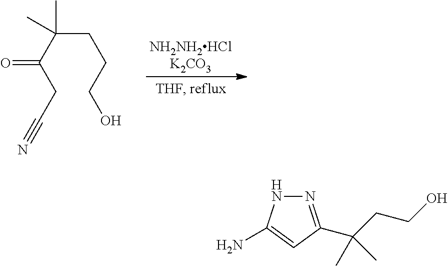

To a solution of 7-hydroxy-4,4-dimethyl-3-oxo-heptanenitrile (0.578 g, 3.42 mmol) in ethanol (6.83 mL) was added hydrazine hydrochloride (0.351 g, 5.12 mmol) followed by K₂CO₃ (0.708 g, 5.12 mmol). The reaction mixture was refluxed under N₂ overnight. The reaction was concentrated in vacuo. The residue was purified by column chromatography (gradient of MeOH in DCM). (300 mg). ¹H NMR (CDCl₃) 7.43 (br s, 1H); 5.43 (s, 1H); 3.85 (br s, 2H); 3.62-3.54 (m, 2H); 1.69-1.60 (m, 2H); 1.54-1.38 (m, 2H); 1.26 (s, 3H); 1.18 (s, 3H).

### Preparation of 4,4-dimethyl-6,7-dihydro-5H-pyrazolo[1,5-a]pyridin-2-amine (Step 5)

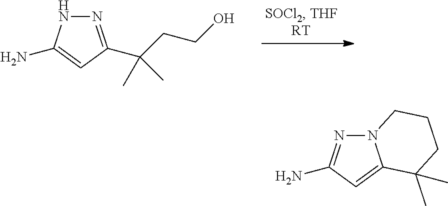

To a solution of 3-(5-amino-1H-pyrazol-3-yl)-3-methyl-butan-1-ol (0.300 g, 1.64 mmol) in tetrahydrofuran (9.82 mL) at rt was added thionyl chloride (0.597 mL, 8.19 mmol). The reaction mixture was stirred at rt for 2 h and checked by LCMS. Only the cyclic material was detected. The reaction was then quenched with water and extracted with EtOAc. The combined organic layer was washed with brine, then dried over MgSO₄ and concentrated. ¹H NMR (CDCl₃) (142 mg). 5.38 (s, 1H); 3.88 (t, 2H); 3.63 (br s, 2H); 2.06-1.98 (m, 2H); 1.68-1.61 (m, 2H); 1.25 (s, 6H).

### Example 4

### Preparation of 1-ethyl-6,6-dimethyl-4,5-dihydrocyclopenta[c]pyrazol-3-amine

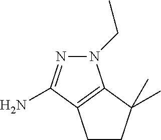

### Preparation of ethyl 2-(3,3-dimethyl-2-oxo-cyclopentyl)-2-oxo-acetate (Step 1)

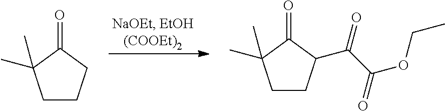

To a solution of sodium ethoxide (3 g, 44.1 mmol) in ethanol (15 ml) at −15° C. under N₂ was added slowly a solution of 2,2-dimethylcyclopentanone (5 g, 44.6 mmol) and diethyl oxalate (6.514192 g, 6.048 mL, 44.6 mmol) in ethanol (15 mL). The resulting mixture was stirred at −15° C. for 15 min and then the cold bath was removed. The reaction was then stirred at rt overnight. The reaction was quenched with HCl. It was then extracted with DCM. The combined chlorinated layers were washed with water, dried over MgSO₄ and concentrated. (8.2 g)¹H NMR (CDCl₃) 4.36 (q, 2H); 2.88 (t, 2H); 1.81 (t, 2H); 1.41 (t, 3H); 1.16 (s, 6H).

### Preparation of 1-ethyl-3,6,6-trimethyl-4,5-dihydrocyclopenta[c]pyrazole (Step 2)

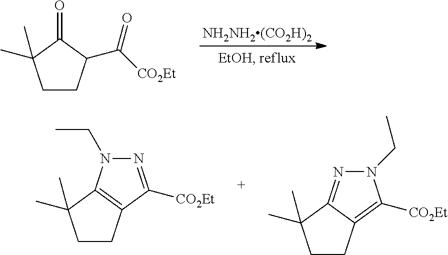

To a solution of ethyl 2-(3,3-dimethyl-2-oxo-cyclopentyl)-2-oxo-acetate (7.7 g, 36.3 mmol) in ethanol (150 ml) was added ethylhydrazine oxalate (6.0 g, 40 mmol). The resulting mixture was heated at reflux for 3.5 h. It was then cooled down and concentrated in vacuo. The residue was taken into DCM and washed with NaHCO₃. The chlorinated layer was then dried over MgSO4 and concentrated. The residue was then purified by column chromatography (gradient of EtOAc in hexane)

Pure regioisomer 1-Et-5-COOEt: (1.9 g). ¹H NMR (CDCl₃) 4.53 (q, 2H); 4.33 (q, 2H); 2.76 (m, 2H); 2.21 (m, 3H); 1.38 (t, 3H); 1.34 (t, 3H); 1.21 (s, 6H)

Pure regioisomer 1-Et-3-COOEt: (5.2 g). ¹H NMR (CDCl₃) 4.38 (q, 2H); 4.18 (q, 2H); 2.73 (t, 2H); 2.38 (t, 3H); 1.48 (t, 3H); 1.38 (t, 3H); 1.33 (s, 6H).

### Preparation of 1-ethyl-6,6-dimethyl-4,5-dihydrocyclopenta[c]pyrazole-3-carboxylic acid (Step 3)

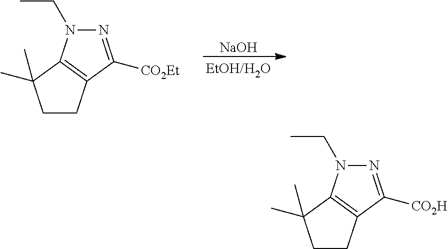 [SMILES: CCOC(=O)c1nn(CC)c2c1CCC2(C)C ; "NaOH" ; CCn1nc(C(=O)O)c2c1C(C)(C)CC2]

To a solution of pyrazole-3-ethyl-carboxylic ester (5.2 g, 22 mmol) in ethanol (150 ml) was added a 2N solution of sodium hydroxide (25 ml). The resulting mixture was stirred at rt for 7 h. It was concentrated in vacuo and the residue was taken into water. It was then cooled into an ice bath and acidified with 2N HCl. A white solid precipitated out and was filtered off, washed with water and air dried. ¹H NMR (CDCl₃) (4.58 g) 4.13 (q, 2H); 2.76 (t, 2H); 2.39 (t, 3H); 1.48 (t, 3H); 1.38 (s, 6H).

### Preparation of 1-ethyl-6,6-dimethyl-4,5-dihydrocyclopenta[c]pyrazol-3-amine (Step 4)

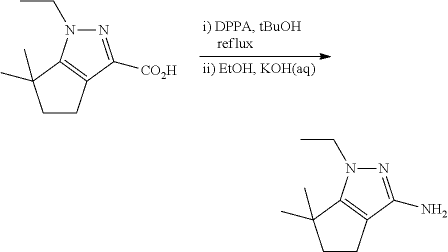

To a suspension of 1-ethyl-6,6-dimethyl-4,5-dihydrocyclopenta[c]pyrazole-3-carboxylic acid (625 mg, 3 mmol) in t-BuOH (15 mL) was added DPPA (0.712 mL, 3.3 mmol) and then Et₃N (0.250 mL, 3.6 mmol). The reaction mixture was heated to reflux. No exotherm was noticed (or controlled by reflux) and a small amount of N₂ bubbled away. After several LCMS checks, the reaction was heated at reflux overnight. The reaction was then checked by LCMS. The reaction was quenched with K₂CO₃ and extracted with EtOAc. The combined organic layers were washed with brine, then dried over MgSO₄ and concentrated. (1.526 g) (contained (PhO)₂POOH). ¹H NMR (CDCl₃) 3.94 (q, 2H); 2.66 (br t, 2H); 2.29 (t, 2H); 1.40 (t, 3H); 1.31 (s, 6H).

To a solution of the crude urea, from above, (192 mg, 0.5 mmol) in ethanol (0.2 mL) at rt was added a solution of potassium hydroxide 20 wt % in water (2 mL) in a microwave vial. The vial was then was irradiated at 160° C. for 30 min, generating 15-20 bars of pressure. The reaction was then checked by LCMS, quenched with water and extracted with EtOAc. The combined organics were washed with brine, dried over MgSO₄ and concentrated.

(65 mg). ¹H NMR (CDCl₃) 3.85 (q, 2H); 3.45 (br s, 2H); 2.41 (t, 2H); 2.27 (t, 2H); 1.38 (t, 3H); 1.29 (s, 6H).

### Example 5

### Preparation of 3-chloro-2-hydroxy-1-(5-iodo-1-methyl-pyrazol-3-yl)-4-methyl-2H-pyrrol-5-one (B8)

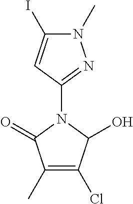 [SMILES: CC1=C(Cl)C(O)N(c2cc(I)n(C)n2)C1=O]

### Preparation of 3-(2,5-dimethylpyrrol-1-yl)-5-iodo-1-methyl-pyrazole (Step 1)

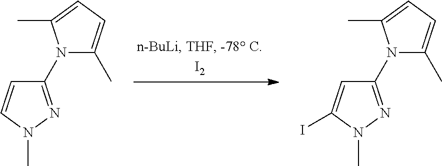

A stirred solution of 3-(2,5-dimethylpyrrol-1-yl)-1-methyl-pyrazole (2.500 g, 14.27 mmol) in tetrahydrofuran (40 mL) was cooled to −78° C. under an atmosphere of nitrogen. n-Butyllithium (1.6 mol in hexanes) (9.8 mL, 15.69 mmol) was added over 15 mins. The resulting solution was stirred at −78° C. for 2 hours. Iodine (3.621 g, 14.27 mmol) was dissolved in THF (10 mL) and added over 10 mins, maintaining a temperature below −60 C. The reaction mixture was stirred cold for a further 10 minutes and then allowed to warm slowly to ambient temperature over a period of 1 hour. 2M Hydrochloric acid was added to quench the reaction. Dichloromethane was added and the layers separated. The aqueous layer was extracted three times with dichloromethane. The combined organics washed with saturated aqueous sodium metabisulfite, dried over MgSO₄ and concentrated in vacuo to brown oil. Chromatography on silica gel gave a white solid (2.952 g, 69%). ¹H NMR (CDCl₃) δ 6.34 (1H, s), 5.86 (2H, s), 3.96 (3H, s), 2.11 (6H, s).

### Preparation of 5-iodo-1-methyl-pyrazol-3-amine (Step 2)

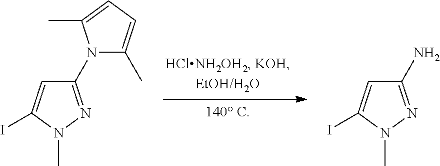

To a stirred solution of 3-(2,5-dimethylpyrrol-1-yl)-5-iodo-1-methyl-pyrazole (1.00 g, 3.32 mmol) and hydroxylamine hydrochloride (1.17 g, 16.6 mmol) in ethanol (10 mL), was added potassium hydroxide (0.466 g, 8.30 mmol) dissolved in water (5 mL). The solution was heated with stirring at 140° C. for one hour under microwave irradiation. Water and dichloromethane were added and the layers separated. The aqueous layer was extracted three times with dichloromethane. The combined organics washed with water, dried over MgSO₄ and concentrated in vacuo to a brown oil. Chromatography on silica gel gave an orange solid (0.348 g, 47%). ¹H NMR (CDCl₃) δ 5.77 (s, 1H), 3.74 (s, 3H), 3.63 (br. s, 2H).

### Preparation of 3-chloro-1-(5-iodo-1-methyl-pyrazol-3-yl)-4-methyl-pyrrole-2,5-dione (Step 3)

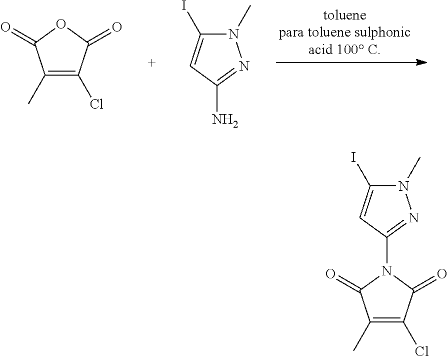

To a stirred solution of 5-iodo-1-methyl-pyrazol-3-amine (0.878 g, 3.94 mmol) and p-toluene sulphonic acid (0.136 g, 0.79 mmol) in toluene (40 mL), was added 3-chloro-4-methyl-furan-2,5-dione (0.635 g, 4.33 mmol.) The solution was heated at 110° C. for one hour and then allowed to cool to ambient temperature. The solution was then concentrated in vacuo to brown oil. Chromatography on silica gel gave an orange solid (0.890 g, 64%). ¹H NMR (CDCl₃) δ 6.49 (s, 1H), 3.96 (s, 3H), 2.15 (s, 3H).

### Preparation of 3-chloro-2-hydroxy-1-(5-iodo-1-methyl-pyrazol-3-yl)-4-methyl-2H-pyrrol-5-one (B8) and 4-chloro-2-hydroxy-1-(5-iodo-1-methyl-pyrazol-3-yl)-3-methyl-2H-pyrrol-5-one (Step 4)

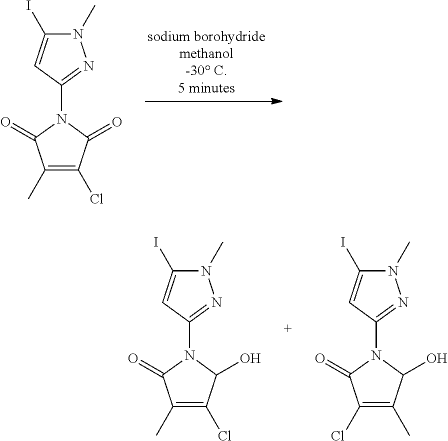 [SMILES: CC1=C(Cl)C(=O)N(c2cc(I)n(C)n2)C1=O ; CC1=C(Cl)C(O)N(c2cc(I)n(C)n2)C1=O ; CC1=C(Cl)C(=O)N(c2cc(I)n(C)n2)C1O]

To 3-chloro-1-(5-iodo-1-methyl-pyrazol-3-yl)-4-methyl-pyrrole-2,5-dione (0.850 g, 2.42 mmol) in methanol (10 mL) and tetrahydrofuran (5 mL) at −30° C., was added sodium borohydride (0.103 g, 2.66 mmol). The solution was stirred for one hour. Water and ethyl acetate were added and the layers separated. The aqueous layer was extracted three times with ethyl acetate. The combined organics were washed with water, dried over MgSO₄ and concentrated in vacuo to afford a white solid. Chromatography on silica gel gave 3-chloro-2-hydroxy-1-(5-iodo-1-methyl-pyrazol-3-yl)-4-methyl-2H-pyrrol-5-one (B8) (0.220 g, 26%) and 4-chloro-2-hydroxy-1-(5-iodo-1-methyl-pyrazol-3-yl)-3-methyl-2H-pyrrol-5-one (0.459 g, 54%). 3-chloro-2-hydroxy-1-(5-iodo-1-methyl-pyrazol-3-yl)-4-methyl-2H-pyrrol-5-one ¹H NMR (CDCl₃) δ 6.89 (s, 1H), 5.87 (m, 1H), 4.54 (d, 1H), 3.86 (s, 3H), 1.96 (s, 3H). 4-chloro-2-hydroxy-1-(5-iodo-1-methyl-pyrazol-3-yl)-3-methyl-2H-pyrrol-5-one ¹H NMR (CDCl₃) δ 6.93 (s, 1H), 5.86 (d, 1H), 4.56 (d, 1H), 3.86 (s, 3H), 2.15 (s, 3H).

### Example 6

### Preparation of 3-chloro-2-hydroxy-4-methyl-1-[1-methyl-5-(2-methylprop-1-enyl)pyrazol-3-yl]-2H-pyrrol-5-one (B7)

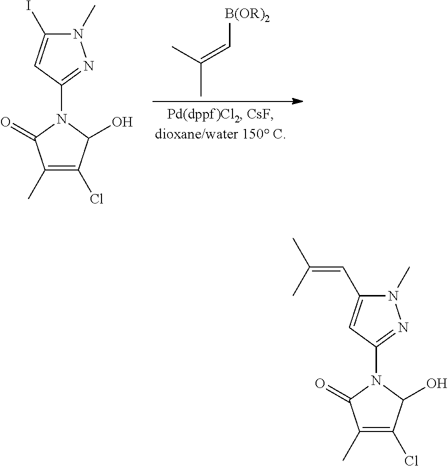

3-chloro-2-hydroxy-1-(5-iodo-1-methyl-pyrazol-3-yl)-4-methyl-2H-pyrrol-5-one (0.225 g, 0.64 mmol), 4,4,5,5-tetramethyl-2-(2-methylprop-1-enyl)-1,3,2-dioxaborolane (0.116 g, 0.64 mmol), cesium fluoride (0.193 g, 1.27 mmol) and [1,1′-Bis(diphenylphosphino)ferrocene] dichloropalladium (II) (0.023 g, 0.032 mmol) combined in 1,4-dioxane (3 mL) and water (1 mL). The stirred solution was heated to 150° C. for 20 minutes under microwave irradiation. Water and dichloromethane were added and the layers separated. The aqueous layer was extracted three times with dichloromethane. The combined organics washed with water, dried over MgSO₄ and concentrated in vacuo to afford a brown oil. Chromatography on silica gel gave a white solid (0.094 g, 52%). ¹H NMR (CDCl₃) δ 6.62 (s, 1H), 5.97 (m, 1H), 5.89 (m, 1H), 4.93 (d, 1H), 3.70 (s, 3H), 1.96 (s, 6H), 1.90 (s, 3H).

### Example 7

### Preparation of 3-chloro-2-hydroxy-4-methyl-1-[1-methyl-5-(3-methylbut-2-enyl)pyrazol-3-yl]-2H-pyrrol-5-one (B10)

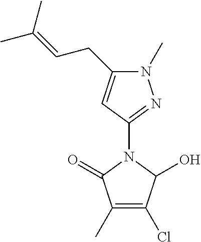 [SMILES: CC(C)=CCc1cc(N2C(=O)C(C)=C(Cl)C2O)nn1C]

### Preparation of 3-(2,5-dimethylpyrrol-1-yl)-1-methyl-5-(3-methylbut-2-enyl)pyrazole (Step 1)

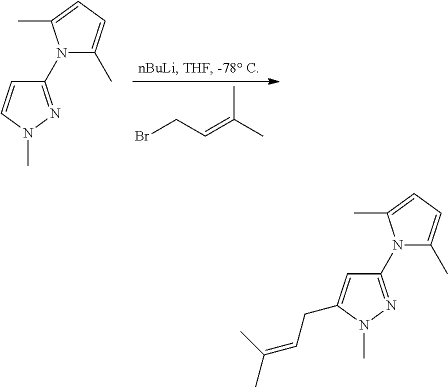

A stirred solution of 3-(2,5-dimethylpyrrol-1-yl)-1-methyl-pyrazole (2.000 g, 11.41 mmol) in tetrahydrofuran (15 mL) was cooled to −78° C. under an atmosphere of nitrogen. Butyllithium (1.6 mol in hexanes) (7.8 mL, 12.55 mmol) was then added over 15 mins. The resulting solution was stirred at −78° C. for 2 hours. 1-bromo-3-methyl-but-2-ene (1.701 g, 11.41 mmol) dissolved in THF (5 mL) was then added over 10 mins, maintaining a temperature below −60° C. The reaction mixture was stirred cold for a further 10 minutes and then allowed to warm slowly to ambient temperature over a period of 1 hour. Saturated aqueous ammonium chloride was added to quench the reaction. Ethyl acetate was then added and the layers separated. The aqueous layer was extracted three times with ethyl acetate. The combined organics washed with water, dried over MgSO₄ and concentrated in vacuo to brown oil. Chromatography on silica gel gave yellow oil 1.728 g (62%). ¹H NMR (CDCl₃) δ 5.90 (s, 1H), 5.83 (s, 2H), 5.30-5.25 (m, 1H), 3.79 (s, 3H), 3.36-3.32 (m, 2H), 2.11 (s, 6H), 1.78 (s, 3H), 1.73 (s, 3H).

### Preparation of 1-methyl-5-(3-methylbut-2-enyl)pyrazol-3-amine (Step 2)

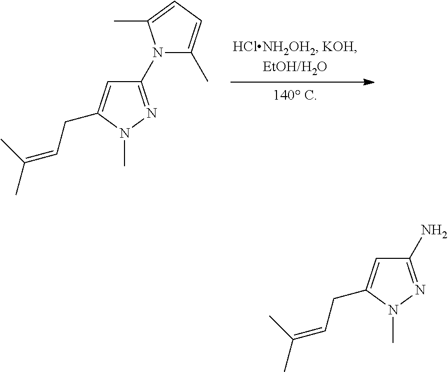 [SMILES: CC(C)=CCc1cc(-n2c(C)ccc2C)nn1C ; CC(C)=CCc1cc(N)nn1C]

To a stirred solution of 3-(2,5-dimethylpyrrol-1-yl)-1-methyl-5-(3-methylbut-2-enyl)pyrazole (1.316 g, 5.41 mmol) and hydroxylamine hydrochloride (1.898 g, 27.03 mmol,) in ethanol (10 mL), was added potassium hydroxide (0.758 g, 13.52 mmol) dissolved in water (5 mL). The solution was heated with stirring at 140° C. for one hour under microwave irradiation. Water and dichloromethane were added and the layers separated. The aqueous layer was extracted three times with dichloromethane. The combined organics washed with water, dried over MgSO₄ and concentrated in vacuo to orange oil. Chromatography on silica gel gave yellow oil (0.642 g, 71%) ¹H NMR (CDCl₃) δ 5.38 (s, 1H), 5.24-5.18 (m, 1H), 3.58 (s, 3H), 3.55-3.46 (br. s, 2H), 3.20-3.17 (m, 2H), 1.75 (s, 3H), 1.69 (s, 3H)

### Preparation of 3-chloro-4-methyl-1-[1-methyl-5-(3-methylbut-2-enyl)pyrazol-3-yl]pyrrole-2,5-dione (Step 3)

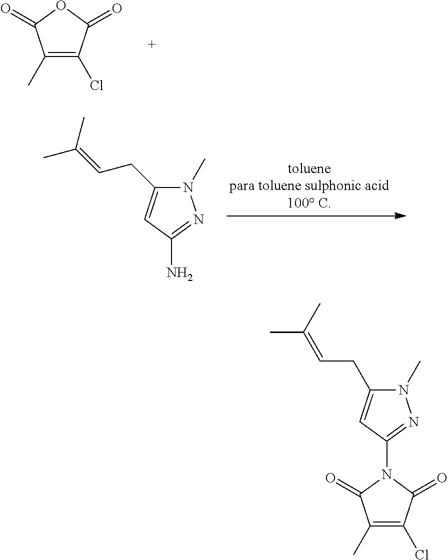

To a stirred solution of 1-methyl-5-(3-methylbut-2-enyl)pyrazol-3-amine (0.321 g, 1.94 mmol) and p-toluene sulphonic acid (0.067 g, 0.39 mmol) in toluene (10 mL) was added 3-chloro-4-methyl-furan-2,5-dione (0.285 g, 1.94 mmol.) The solution was heated at 85° C. for two hours and then allowed to cool to ambient temperature. The solution was then concentrated in vacuo to an orange oil. Chromatography on silica gel gave a yellow solid (0.399 g, 70%). ¹H NMR (CDCl₃) 6.05 (s, 1H), 5.27-5.23 (m, 1H), 3.79 (s, 3H), 3.33-3.29 (m, 2H), 2.14 (s, 3H), 1.76 (s, 3H), 1.70 (s, 3H).

### Preparation of 3-chloro-2-hydroxy-4-methyl-1-[1-methyl-5-(3-methylbut-2-enyl)pyrazol-3-yl]-2H-pyrrol-5-one (B10) and 4-chloro-2-hydroxy-3-methyl-1-[1-methyl-5-(3-methylbut-2-enyl)pyrazol-3-yl]-2H-pyrrol-5-one (Step 4)

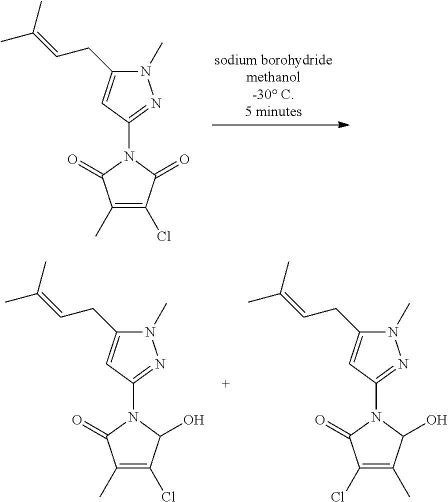 [SMILES: CC(C)=CCc1cc(N2C(=O)C(C)=C(Cl)C2=O)nn1C ; CC(C)=CCc1cc(N2C(=O)C(C)=C(Cl)C2O)nn1C ; CC(C)=CCc1cc(N2C(=O)C(Cl)=C(C)C2O)nn1C]

To 3-chloro-4-methyl-1-[1-methyl-5-(3-methylbut-2-enyl)pyrazol-3-yl]pyrrole-2,5-dione (0.374 g, 1.27 mmol) in methanol (10 mL) and tetrahydrofuran (5 mL) at −15° C., was added sodium borohydride (0.054 g, 1.40 mmol). The solution was stirred for two hours. Water and dichloromethane were added and the layers separated. The aqueous layer was extracted three times with dichloromethane. The combined organics washed with water, dried over MgSO₄ and concentrated in vacuo to afford a colourless oil. Chromatography on silica gel gave 3-chloro-2-hydroxy-4-methyl-1-[1-methyl-5-(3-methylbut-2-enyl)pyrazol-3-yl]-2H-pyrrol-5-one (B10) (0.087 g, 23%) and 4-chloro-2-hydroxy-3-methyl-1-[1-methyl-5-(3-methylbut-2-enyl)pyrazol-3-yl]-2H-pyrrol-5-one (0.171 g, 45%). 3-chloro-2-hydroxy-4-methyl-1-[1-methyl-5-(3-methylbut-2-enyl)pyrazol-3-yl]-2H-pyrrol-5-one ¹H NMR (CDCl₃) δ 6.45 (s, 1H), 5.87-5.85 (m, 1H), 5.26-5.20 (m, 1H), 4.92 (d, 1H), 3.69 (s, 3H), 3.31-3.25 (m, 2H), 1.95 (s, 3H), 1.79 (s, 3H), 1.71 (s, 3H). 4-chloro-2-hydroxy-3-methyl-1-[1-methyl-5-(3-methylbut-2-enyl)pyrazol-3-yl]-2H-pyrrol-5-one ¹H NMR (CDCl₃) δ 6.49 (s, 1H), 5.86-5.84 (m, 1H), 5.26-5.20 (m, 1H), 4.94 (d, 1H), 3.69 (s, 3H), 3.30-3.27 (m, 2H), 2.14 (s, 3H), 1.76 (s, 3H), 1.71 (s, 3H)

### Example 8

### Preparation of 3-amino-5-tert-butyl-1-methyl-pyrazole-4-carbonitrile

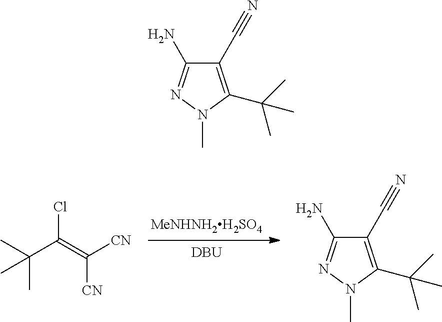

To a 3 neck round bottom flask, DBU (3 equiv., 3.5583 mmol) and methyl hydrazine sulphate (1.2 equiv., 1.4233 mmol) was added into 2-methyl-2-propanol (10 mL/g, 21.2 mmol) at RT under Nitrogen atmosphere and stirred for 60 min at RT. 2-(1-chloro-2,2-dimethyl-propylidene)propanedinitrile (200 mg, 1.1861 mmol) was added in minimum amount of 2-methyl-2-propanol (10 mL/g, 21.2 mmol) and the mixture was stirred for 2 h at RT. After this time, the reaction mixture was quenched with water (20 ml) and extracted with ethyl acetate (3×50 ml) and the combined organic extracts were dried over Na₂SO₄ and concentrated under reduced pressure. The desired compound was purified by column chromatography (442 mg, 70%).

### Example 9

### Preparation of 1-(5-tert-butyl-1-methyl-pyrazol-3-yl)-3-chloro-2-hydroxy-4-methoxy-2H-pyrrol-5-one (J1)

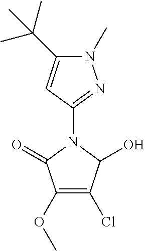 [SMILES: COC1=C(Cl)C(O)N(c2cc(C(C)(C)C)n(C)n2)C1=O]

### Step 1 Preparation of 1-(5-tert-butyl-1-methyl-pyrazol-3-yl)-3-chloro-4-hydroxy-2H-pyrrol-5-one

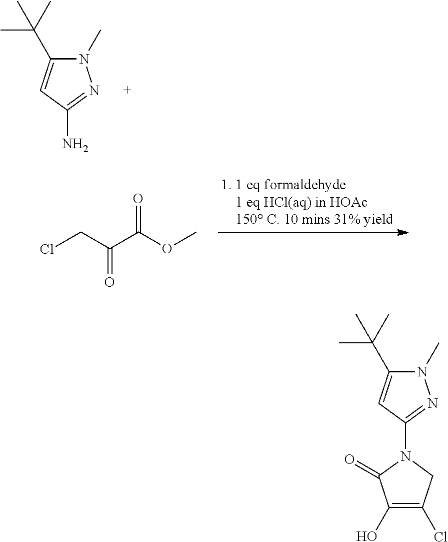

Methyl 3-chloro-2-oxo-propanoate (510 mg, 3.80 mM) was dissolved in acetic acid (2.5 ml), then 5-tert-butyl-1-methyl-pyrazol-3-amine (291 mg, 1.90 mM) was added, followed by formaldehyde solution (37% aqueous, 0.156 ml, 2.09 mM). Finally, concentrated HCl (37% aq, 0.180 ml) was added and the reaction was heated to 150° C. in a microwave for 10 mins. The reaction was poured into water (30 ml) and extracted with ethyl acetate (20 ml×3) and the combined extracts were dried over sodium sulphate, filtered and evaporated to give a dark brown gum (1.00 g), which was chromatographed to give a pale yellow gum (200 mg, 31% yield).

1H NMR (d6DMSO) 10.6 (br s, 1H), 6.37 (s, 1H), 4.33 (s, 2H), 3.83 (s, 3H), 1.34 (s, 9H).

### Step 2 Preparation of 1-(5-tert-butyl-1-methyl-pyrazol-3-yl)-3-chloro-4-methoxy-2H-pyrrol-5-one

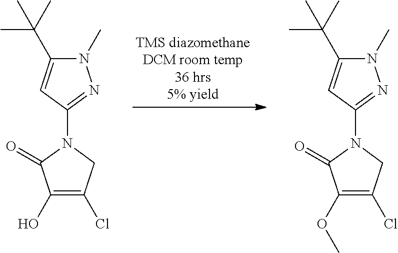 [SMILES: Cn1nc(N2CC(Cl)=C(O)C2=O)cc1C(C)(C)C ; COC1=C(Cl)CN(c2cc(C(C)(C)C)n(C)n2)C1=O]

1-(5-tert-butyl-1-methyl-pyrazol-3-yl)-3-chloro-4-hydroxy-2H-pyrrol-5-one (2.00 g, 7.41 mM) was dissolved in dichloromethane (45 ml) then trimethylsilyl diazomethane (2M in diethyl ether, 4.5 ml, 8.89 mM) was added dropwise slowly, effervescence, over 1 hr. The reaction was stirred at room temperature for 18 hrs. A further portion of trimethylsilyl diazomethane (2M in diethyl ether, 1.12 ml, 2.24 mM) was added dropwise, slowly over 20 mins with stirring at room temperature. After 18 hrs acetic acid (2 ml) was added dropwise to destroy any excess TMS diazomethane, this caused effervescence. The mixture was stirred at room temperature for 1 hr, then the reaction was then evaporated to give a black oil (2.23 g) which was chromatographed to give a pale yellow solid (117 mg, 5% yield).

1H NMR (CDCl3) 6.54 (s, 1H), 4.33 (s, 2H), 4.16 (s, 3H), 3.88 (s, 3H), 1.38 (s, 9H).

### Step 3 Preparation of [1-(5-tert-butyl-1-methyl-pyrazol-3-yl)-3-chloro-4-methoxy-5-oxo-2H-pyrrol-2-yl] acetate

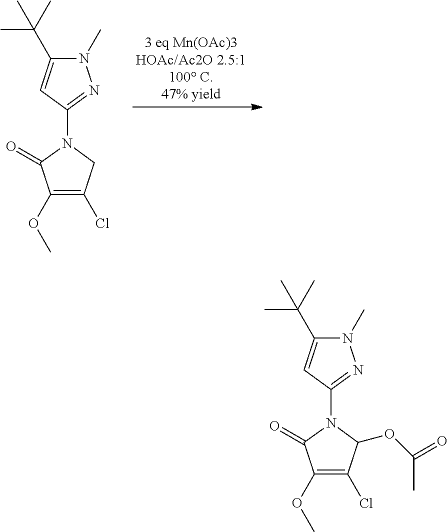 [SMILES: COC1=C(Cl)CN(c2cc(C(C)(C)C)n(C)n2)C1=O ; COC1=C(Cl)C(OC(C)=O)N(c2cc(C(C)(C)C)n(C)n2)C1=O]

1-(5-tert-butyl-1-methyl-pyrazol-3-yl)-3-chloro-4-methoxy-2H-pyrrol-5-one (112 mg, 0.395 mM) was dissolved in acetic acid (1.12 ml) and acetic anhydride (0.448 ml) then manganese triacetate dihydrate (317 mg, 1.184 mM) were added to give a paste. The reaction was then heated to 100° C. in a microwave for 15 mins. Diethyl ether (10 ml) and water (7 ml) were added, and the reaction shaken. The layers were separated and the aqueous layer was extracted with ether (2×10 ml). The combined ether layers were dried over Na2SO4, filtered and evaporated to give an amber gum which was purified by chromatography to give a pale amber gum (64 mg, 47% yield).

1H NMR (CDCl3) 7.05 (s, 1H), 6.39 (s, 1H), 4.21 (s, 3H), 3.33 (s, 3H), 2.16 (s, 3H), 1.37 (s, 9H).

### Step 4 Preparation of 1-(5-tert-butyl-1-methyl-pyrazol-3-yl)-3-chloro-2-hydroxy-4-methoxy-2H-pyrrol-5-one

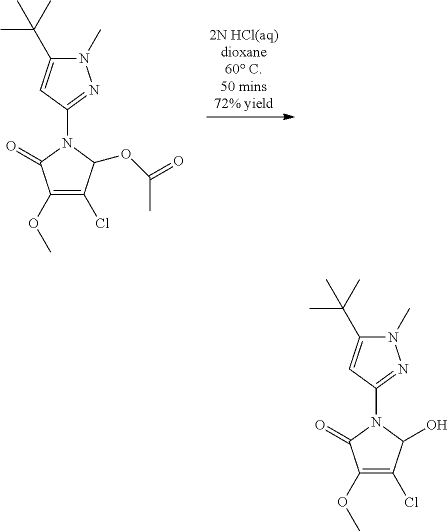 [SMILES: COC1=C(Cl)C(OC(C)=O)N(c2cc(C(C)(C)C)n(C)n2)C1=O ; COC1=C(Cl)C(O)N(c2cc(C(C)(C)C)n(C)n2)C1=O]

[1-(5-tert-butyl-1-methyl-pyrazol-3-yl)-3-chloro-4-methoxy-5-oxo-2H-pyrrol-2-yl] acetate (49 mg, 0.143 mM) was dissolved in 1,4 dioxane (0.60 ml) then HCl (2M aqueous, 0.60 ml, 1.20 mM) was added and the reaction was heated to 60° C. in a microwave for 50 minutes. The reaction was diluted with ethyl acetate (5 ml), water (1 ml) and brine (aqueous, 0.3 ml), shaken and separated. The aqueous phase was further extracted with ethyl acetate (2×3 ml) and the combined extracts were dried over Na2SO4, filtered and evaporated to give a gum which was chromatographed to give 32 mg crystalline white solid (32 mg, 72% yield).

NMR 1H data (CDCl3) 6.46 (s, 1H), 5.81 (m, 1H), 4.97 (br m, 1H), 4.21 (s, 3H), 3.87 (s, 3H), 1.38 (s, 9H).

Table 1 lists compounds of the general formula

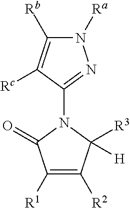

wherein R¹, R², R³, R^{a}, R^{b} and R^{c} are as defined in the table.

These compounds were made by the general methods of Examples 1 to 7. Intermediates were prepared as described in Schemes 1 to 12 or as described in the literature. It is noted that, when R^{a} and R^{b} or R^{b} and R^{c} form a ring structure, the whole of the substituted pyrazole ring is shown in the table for clarity.

TABLE 1
Compounds of the Invention

| | | | | | | | 1H NMR |
|---|---|---|---|---|---|---|---|
| | | | | | | | (measured in |
| | | | | | | | CDCl₃ unless |
| Compound | | | | | | | otherwise |
| Number | R¹ | R² | R³ | R^{a} | R^{b} | R^{c} | indicated) δ |
| A1 | Me | Cl | OH | H | tBu | H | 6.59 (s, 1H); 5.92 |
| | | | | | | | (br d, 1H); 1.97 (d, |
| | | | | | | | 3H); 1.36 (s, 9H) - |
| | | | | | | | no OH or NH |
| | | | | | | | detected. |
| A2 | Me | Cl | OH | H | CF3 | H | 6.50 (br s, 1H); |
| | | | | | | | 5.63 (br s, 1H); |
| | | | | | | | 2.95 (br s, 2H); |
| | | | | | | | 1.92 (s, 3H) |
| A3 | Me | Cl | OH | H | iPr | H | 6.30 (br s, 1H); |
| | | | | | | | 5.71 (br s, 1H); |
| | | | | | | | 2.86 (hept, 1H); |
| | | | | | | | 1.82 (d, 3H); 1.17 |
| | | | | | | | (d, 6H) - no OH or |
| | | | | | | | NH detected |
| A4 | Me | Cl | OH | H | cyPr | H | 6.18 (br s, 1H); |
| | | | | | | | 5.81 (br s, 1H); |
| | | | | | | | 1.91-1.95 (m, 4H); |
| | | | | | | | 0.98-1.04 (m, 2H); |
| | | | | | | | 0.73-0.78 (m, 2H) - |
| | | | | | | | in CD3OD, no OH |
| | | | | | | | or NH detected |
| A5 | Me | Cl | OH | H | Me | H | 6.31 (br s, 1H); |
| | | | | | | | 5.82 (br s, 1H); |
| | | | | | | | 2.31 (s, 3H); 1.93 |
| | | | | | | | (s, 3H); - in |
| | | | | | | | CD3OD, no OH or |
| | | | | | | | NH detected |
| A6 | Me | Cl | OH | H | CF2CF3 | H | 6.58 (br s, 1H); |
| | | | | | | | 5.64 (br s, 1H); |
| | | | | | | | 1.91 (s, 3H) - no |
| | | | | | | | OH or NH in CDCl3 |
| | | | | | | | + a few drops of |
| | | | | | | | CD3OD |
| A7 | Me | Cl | OH | H | CMe(Et)2 | H | 5.93 (s, 1H), 1.96 |
| | | | | | | | (s, 3H), 1.66 (m, |
| | | | | | | | 2H), 1.61 (m, 2H), |
| | | | | | | | 1.26 (s, 3H), 0.78 |
| | | | | | | | (t, 6H). |
| B1 | Me | Cl | OH | Me | Me | H | 6.28 (s, 1H); 5.78 |
| | | | | | | | (pseudo q, 1H); |
| | | | | | | | 3.72 (s, 3H); 2.27 |
| | | | | | | | (s, 3H); 1.89 (d, 3H) |
| | | | | | | | - in CD3OD, no OH |
| | | | | | | | detected |
| B2 | Me | Cl | OH | Me | tBu | H | 6.49 (s, 1H), 5.86 |
| | | | | | | | (s, 1H), 5.05 (bs, |
| | | | | | | | 1H), 3.87 (s, 3H), |
| | | | | | | | 1.94 (d, 3H), 1.39 |
| | | | | | | | (s, 9H). |
| B3 | Me | Cl | OH | Me | CF3 | H | 7.06 (s, 1H); 5.85 |
| | | | | | | | (br s, 1H); 4.51 (br |
| | | | | | | | s, 1H); 3.92 (s, 3H); |
| | | | | | | | 1.96 (d, 3H) |
| B4 | Me | Cl | OH | Me | CF2CF3 | H | 7.10 (s, 1H); 5.92 |
| | | | | | | | (br d, 1H); 4.35 (d, |
| | | | | | | | 1H); 3.94 (s, 3H); |
| | | | | | | | 1.97 (d, 3H) |
| B5 | Me | Cl | OH | Me | iPr | H | 6.48 (s, 1H), 5.86 |
| | | | | | | | (s, 1H), 5.08 (s, 1H), |
| | | | | | | | 3.71 (s, 3H), 2.9 |
| | | | | | | | (m, 1H), 1.94 (s, 3H), |
| | | | | | | | m 1.25 (d, 6H) |
| B6 | Me | Cl | OH | Me | C(CH3)2CH2CH═CH2 | H | 6.49 (s, 1H), 5.87 |
| | | | | | | | (m, 1H), 5.59 (m, |
| | | | | | | | 1H), 5.07 (m, 1H), |
| | | | | | | | (d, 1H), 3.87 (s, |
| | | | | | | | 3H), 2.44 (d, 2H), |
| | | | | | | | 1.95 (s, 3H), 1.37 |
| | | | | | | | (s, 6H) |
| B7 | Me | Cl | OH | Me | CH═C(CH3)2 | H | 6.62 (s, 1H), 5.97 |
| | | | | | | | (m, 1H), 5.89 (m, |
| | | | | | | | 1H), 4.93 (d, 1H), |
| | | | | | | | 3.70 (s, 3H), 1.96 |
| | | | | | | | (s, 6H), 1.90 (s, 3H) |
| B8 | Me | Cl | OH | Me | I | H | 6.89 (s, 1H), 5.87 |
| | | | | | | | (m, 1H), 4.54 (d, |
| | | | | | | | 1H), 3.86 (s, 3H), |
| | | | | | | | 1.96 (s, 3H). |
| B9 | Me | Cl | OH | Me | Cl | H | 6.69 (s, 1H), 5.88 |
| | | | | | | | (m, 1H), 4.52 (d, |
| | | | | | | | 1H), 3.78 (s, 3H), |
| | | | | | | | 1.96 (s, 3H) |
| B10 | Me | Cl | OH | Me | CH2CH═C(CH3)2 | H | 6.45 (s, 1H), 5.87- |
| | | | | | | | 5.85 (m, 1H), 5.26- |
| | | | | | | | 5.20 (m, 1H), 4.92 |
| | | | | | | | (d, 1H), 3.69 (s, |
| | | | | | | | 3H), 3.31-3.25 (m, |
| | | | | | | | 1.79 (s, 3H), 1.71 |
| | | | | | | | (s, 3H) |
| B11 | Me | Cl | OH | Me | CH═CHC(CH3)3 trans | H | 6.65 (s, 1H), 5.97 |
| | | | | | | | (m, 1H), 5.88 (d, |
| | | | | | | | 1H), 4.92 (d, 1H), |
| | | | | | | | 3.71 (s, 3H), 2.15 |
| | | | | | | | (s, 3H), 1.96 (d, |
| | | | | | | | 3H), 1.90 (d, 3H) |
| B12 | Me | Cl | OH | Me | CH2CH2C(H)(Me)2 | H | 6.47 (s, 1H), 5.86 |
| | | | | | | | (m, 1H), 4.95 (d, |
| | | | | | | | 1H), 3.70 (s, 3H), |
| | | | | | | | 2.59-2.53 (m, 2H), |
| | | | | | | | 1.96 (s, 3H), 1.72- |
| | | | | | | | 1.61 (m, 1H), 1.60- |
| | | | | | | | 1.50 (m, 2H), 0.95 |
| | | | | | | | (d, 6H). |
| B13 | Me | Cl | OH | Me | CMe(Et)(OMe) | H | mixture of |
| | | | | | | | diastereoisomers: |
| | | | | | | | 6.56 (s, 1H), 5.91- |
| | | | | | | | 5.87 (m, 1H), 5.10 |
| | | | | | | | (d, 1H), 3.89 (s, |
| | | | | | | | 3H), 3.08 (s, 1.5H), |
| | | | | | | | 3.07 (s, 1.5H), 1.95 |
| | | | | | | | (s, 3H), 1.93-1.78 |
| | | | | | | | (m, 2H), 1.51 (s, |
| | | | | | | | 3H, Me), 0.92-0.81 |
| | | | | | | | (m, 3H). |
| B14 | Me | Cl | OH | Me | CMe(Et)(OEt) | H | mixture of |
| | | | | | | | diastereoisomers: |
| | | | | | | | 6.57 (s, 1H), 5.88- |
| | | | | | | | 5.84 (m, 1H), 4.97 |
| | | | | | | | (d, 1H), 3.92 (s, |
| | | | | | | | 3H), 3.35-3.26 (m, |
| | | | | | | | 1H), 3.18-3.08 (m, |
| | | | | | | | 1H), 2.14 (s, 3H), |
| | | | | | | | 2.00-1.80 (m, 2H), |
| | | | | | | | 1.53-1.51 (m, 3H), |
| | | | | | | | 1.17 (t, 3H), 0.88- |
| | | | | | | | 0.79 (m, 3H). |
| B15 | Me | Cl | OH | Me | CH(Me)Et) | H | 6.47 (s, 1H), 5.87 |
| | | | | | | | (m, 1H), 4.93 (d, |
| | | | | | | | 1H), 3.73 (s, 3H), |
| | | | | | | | 2.72-2.64 (m, 1H), |
| | | | | | | | 1.96 (s, 3H), 1.71- |
| | | | | | | | 1.57 (m, 2H), 1.27- |
| | | | | | | | 1.23 (m, 3H), 0.94- |
| | | | | | | | 0.88 (m, 3H). |
| B16 | Me | Cl | OH | Me | CH(OEt)(tBu) | H | mixture of |
| | | | | | | | diastereoisomers: |
| | | | | | | | 6.59 (s, 0.5H); 6.58 |
| | | | | | | | (s, 0.5H); 5.88 (br |
| | | | | | | | s, 1H); 4.99 (br d, |
| | | | | | | | 0.5H); 4.92 (br d, |
| | | | | | | | 0.5H); 4.01 (pseudo |
| | | | | | | | d, 1H); 3.81 |
| | | | | | | | (pseudo d, 3H); |
| | | | | | | | 3.44-3.35 (m, 1H); |
| | | | | | | | 3.29-3.21 (m, 1H); |
| | | | | | | | 1.95 (s, 3H); 1.16 |
| | | | | | | | (t, 3H); 0.96 (s, 9H) |
| B17 | Me | Cl | OH | Me | SMe | H | 6.68 (s, 1H), 5.88 |
| | | | | | | | (m, 1H), 4.73 (d, |
| | | | | | | | 1H), 3.78 (s, 3H), |
| | | | | | | | 2.47 (s, 3H), 1.96 |
| | | | | | | | (s, 3H). |
| B18 | Me | Cl | OH | 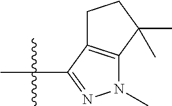 [SMILES: *c1nn(C)c2c1CCC2(C)C] | | | 5.89 (br s, 1H); 5.27 (br s, 1H); 3.69 (s, 3H); 2.92- 2.78 (m, 2H); 2.29 (dt, 2H); 1.93 (s, 3H); 1.36 (s, 3H); 1.35 (s, 3H). |
| B19 | Me | Cl | OH | Me | CH(F)(tBu) | H | mixture of |
| | | | | | | | diastereoisomers: |
| | | | | | | | 6.71 (s, 1H); 5.90- |
| | | | | | | | 5.86 (m, 1H); 5.17 |
| | | | | | | | (d, 1H); 4.81 (d, |
| | | | | | | | 0.5H); 4.75 (d, |
| | | | | | | | 0.5H); 3.81 (s, 3H); |
| | | | | | | | 1.95 (s, 3H); 1.05 |
| | | | | | | | (s, 9H) |
| B20 | Me | Cl | OH | Me | SO2Me | H | 7.30 (s, 1H), 5.92 |
| | | | | | | | (m, 1H), 4.26 (d, |
| | | | | | | | 1H), 4.11 (s, 3H), |
| | | | | | | | 3.21 (s, 3H), 1.98 |
| | | | | | | | (s, 3H). |
| B21 | Me | Cl | OH | Me | CH(Me)(CF3) | H | 6.76 (s, 1H), 5.88 |
| | | | | | | | (m, 1H), 4.74 (d, |
| | | | | | | | 0.5H), 4.66 (d, |
| | | | | | | | 0.5H), 3.79 (s, 3H), |
| | | | | | | | 3.60-3.51 (m, 1H), |
| | | | | | | | 1.96 (s, 3H), 1.57- |
| | | | | | | | 1.53 (m, 3H) |
| B22 | Me | Cl | OH | Me | CMe(F)(tBu) | H | mixture of |
| | | | | | | | diastereoisomers: |
| | | | | | | | 6.47 (s, 1H), 5.88 |
| | | | | | | | (br s, 1H); 4.91 (br |
| | | | | | | | s, 1H); 3.901 |
| | | | | | | | (pseudo d, 3H); |
| | | | | | | | 1.93 (s, 3H); 1.71 |
| | | | | | | | (d, 1H); 1.67-1.62 |
| | | | | | | | (m, 2H); 1.03 |
| | | | | | | | (pseudo d, 9H) |
| B23 | Me | Cl | OH | Me | C(Me)2CN | H | 6.62 (s, 1H), 5.88 |
| | | | | | | | (d, 1H), 4.67 (br, |
| | | | | | | | 1H), 4.0 (s, 3H), |
| | | | | | | | 1.94 (s, 3H), 1.78 |
| | | | | | | | (s, 6H). |
| B24 | Me | Cl | OH | Me | CH═CH(Me) cis | H | 6.72 (s, 1H), 6.22- |
| | | | | | | | 6.17 (m, 1H), 6.04- |
| | | | | | | | 5.95 (m, 1H), 5.91 |
| | | | | | | | (m, 1H), 4.97 (m, |
| | | | | | | | 1H), 3.73 (s, 3H), |
| | | | | | | | 1.96 (s, 3H), 1.92 |
| | | | | | | | (d, 3H) |
| B25 | Me | Cl | OH | Me | (Me)C═CH2 | H | 6.64 (s, 1H), 5.89 |
| | | | | | | | (m, 1H), 5.38 (s, |
| | | | | | | | 1H), 5.17 (s, 1H), |
| | | | | | | | 4.82 (d, 1H), 3.83 |
| | | | | | | | (s, 3H), 2.10 (s, |
| | | | | | | | 3H), 1.96 (s, 3H) |
| B26 | Me | Cl | OH | Me | SOMe | H | 7.08 (s, 1H), 5.91 |
| | | | | | | | (m, 1H), 4.48 (d, |
| | | | | | | | 1H), 4.08 (s, 3H), |
| | | | | | | | 3.03 (s, 3H), 1.97 |
| | | | | | | | (s, 3H) |
| B27 | Me | Cl | OH | Me | (Me)C═CMe2 | H | 6.43 (s, 1H), 5.89 |
| | | | | | | | (d, 1H), 5.05 (m, |
| | | | | | | | 1H), 3.58 (s, 3H), |
| | | | | | | | 1.96 (s, 3H), 1.88 |
| | | | | | | | (s, 3H), 1.84 (s, |
| | | | | | | | 3H), 1.57 (s, 3H) |
| B28 | Me | Cl | OH | 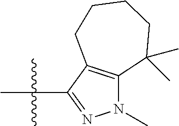 [SMILES: *c1nn(C)c2c1CCCCC2(C)C] | | | 5.57 (br d, 1H); 4.98 (br d, 1H); 3.87 (s, 3H); 2.43- 2.29 (m, 2H); 1.95 (s, 3H); 1.88-1.75 (m, 4H); 1.70-1.61 (m, 2H); 1.48 (s, 3H); 1.45 (s, 3H) |
| B29 | Me | Cl | OH | 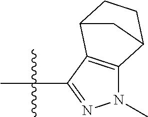 [SMILES: *c1nn(C)c2c1C1CCC2C1] | | | 5.81 (d, 1H); 5.27 (br s, 1H); 3.77 (br d, 1H); 3.68 (s, 3H); 3.32 (br s, 1H); 2.03-1.87 (m, 6H); 1.60 (dd, 1H); 1.45- 1.08 (m, 2H) |
| B30 | Me | Cl | OH | 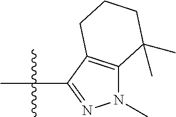 [SMILES: *c1nn(C)c2c1CCCC2(C)C] | | | 5.70 (d, 1H); 5.22 (br s, 1H); 3.80 (s, 3H); 2.54-2.36 (m, 2H); 1.93 (s, 3H); 1.72-1.61 (m, 4H); 1.42 (s, 3H); 1.41 (s, 3H) |
| B31 | Me | Cl | OH | Me | H | H | 7.29 (d, 1H); 6.67 |
| | | | | | | | (d, 1H); 5.88 (m, |
| | | | | | | | 1H); 4.82 (d, 1H); |
| | | | | | | | 3.82 (s, 3H); 1.96 |
| | | | | | | | (s, 3H). |
| B32 | Me | Cl | OH | Me | CH2Ph | H | 7.35-7.23 (m, 3H), |
| | | | | | | | 7.19-7.13 (m, 2H), |
| | | | | | | | 6.53 (s, 1H), 5.88 |
| | | | | | | | (m, 1H), 4.99 (d, |
| | | | | | | | 1H), 3.97 (s, 2H), |
| | | | | | | | 3.61 (s, 3H), 1.95 |
| | | | | | | | (s, 3H). |
| C1 | Me | Cl | OH | Et | CF3 | H | 7.06 (s, 1H); 5.93 |
| | | | | | | | (br d, 1H); 4.44 (d, |
| | | | | | | | 1H); 4.19 (q, 2H); |
| | | | | | | | 1.97 (d, 3H); 1.45 |
| | | | | | | | (t, 3H) |
| C2 | Me | Cl | OH | Et | tBu | H | 6.43 (s, 1H); 5.96 |
| | | | | | | | (m, 1H); 5.02 (d, |
| | | | | | | | 1H); 4.14 (q, 2H); |
| | | | | | | | 1.95 (s, 3H); 1.42 |
| | | | | | | | (t, 3H); 1.37 (s, 9H) |
| C3 | Me | Cl | OH | 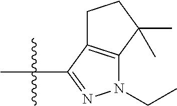 [SMILES: *c1nn(CC)c2c1CCC2(C)C] | | | 5.85 (br d, 1H); 5.23 (d, 1H); 3.95 (q, 2H); 2.92-2.75 (m, 2H); 2.36-2.23 (m, 2H); 1.95 (s, 3H); 1.38 (t, 3H); 1.35 (s, 3H); 1.34 (s, 3H) |
| C4 | Me | Cl | OH | iPr | CF3 | H | 7.04 (s, 1H); 5.95 (br d, 1H); 4.61 (hept, 1H); 4.58 (d, 1H); 1.96 (s, 3H); 1.48 (d, 3H); 1.46 (d, 3H) |
| C5 | Me | Cl | OH | CH2CF3 | CF3 | H | 7.27 (s, 1H); 5.96 |
| | | | | | | | (br d, 1H); 4.72 (q, |
| | | | | | | | 2H); 4.24 (br d, |
| | | | | | | | 1H); 1.97 (s, 3H) |
| C6 | Me | Cl | OH | CF2H | Me | H | 7.08 (t, 1H); 6.71 |
| | | | | | | | (s, 1H); 5.90 (br s, |
| | | | | | | | 1H); 4.41 (d, 1H); |
| | | | | | | | 2.45 (s, 3H); 1.96 |
| | | | | | | | (s, 3H) |
| C7 | Me | Cl | OH | CH2CF3 | CN | H | (DMSO-d6): 7.43 |
| | | | | | | | (s, 1H), 7.12 (d, |
| | | | | | | | 1H), 5.87 (d, 1H), |
| | | | | | | | 5.36 (m, 2H), 1.84 |
| | | | | | | | (s, 3H). |
| C8 | Me | Cl | OH | CH2CF3 | Me | CN | (DMSO-d6): 7.22 |
| | | | | | | | (d, 1H), 5.85 (d, |
| | | | | | | | 1H), 5.23 (m, 2H), |
| | | | | | | | 2.50 (s, 3H), 1.86 |
| | | | | | | | (s, 3H). |
| C9 | Me | Cl | OH | CF3 | Me | H | 6.77 (s, 1H); 5.98 |
| | | | | | | | (br s, 1H), 4.49 (br |
| | | | | | | | s, 1H); 2.44 (s, 3H); |
| | | | | | | | 1.97 (s, 3H). |
| D1 | Me | Cl | OH | Me | tBu | CN | 5.79 (s, 1H), 3.97 |
| | | | | | | | (s, 3H), 1.97 (s, 3H) |
| | | | | | | | 1.55 (s, 9H) |
| D2 | Me | Cl | OH | Me | tBu | Cl | 5.53 (s, 3H), 3.98 |
| | | | | | | | (s, 3H), 1.95 (s, |
| | | | | | | | 3H), 1.52s (s, 9H). |
| D3 | Me | Cl | OH | Me | C(Me)2(CH2Cl) | Cl | 5.60 (s, 1H), 4.03 |
| | | | | | | | (s, 3H), 3.83 (q, |
| | | | | | | | 2H), 1.96 (s, 3H), |
| | | | | | | | 1.65 (s, 6H). |
| D4 | Pr | Me | OH | Me | tBu | CN | |
| E1 | CH2Cl | Cl | OH | Me | tBu | H | 6.51 (s, 1H), 5.96 |
| | | | | | | | (s, 1H), 5.27 9br s, |
| | | | | | | | 1H), 4.32 (d, 2H), |
| | | | | | | | 3.83 (s, 3H), 1.38 |
| | | | | | | | (s, 9H). |
| E2 | nPr | Cl | OH | Me | C(Me)2CN | H | 6.66 (s, 1H), 5.88 |
| | | | | | | | (s, 1H), 4.68 |
| | | | | | | | (br, 1H), 3.99 |
| | | | | | | | (s, 3H), 2.52 (m, 2H), |
| | | | | | | | 1.78 (s, 6H), 1.63 |
| | | | | | | | (m, 2H), 0.98 (t, 3H). |
| F1 | Me | Cl | —OC(O)OCH₂CH(CH₃)₂ | Me | tBu | H | 6.91 (s, 1H), 6.43 |
| | | | | | | | (s, 1H), 4.06 |
| | | | | | | | (quintet, 2H), 3.83 |
| | | | | | | | (s, 3H), 2.05 |
| | | | | | | | (septet, 1H), 1.96 |
| | | | | | | | (s, 3H), 1.36 (s, |
| | | | | | | | 9H), 0.98 (d, 6H) |
| F2 | Me | Cl | —OC(O)OC₆H₅ | Me | tBu | H | 7.41 (m, 2H), 7.25 |
| | | | | | | | (m, 3H), 6.98 (s, |
| | | | | | | | 1H), 6.46 (s, 1H), |
| | | | | | | | 3.87 (s, 3H), 1.99 |
| | | | | | | | (s, 3H), 1.38 (s, |
| | | | | | | | 9H) |
| F3 | OMe | Cl | OC(O)Me | Me | tBu | H | 7.05 (s, 1H), 6.39 |
| | | | | | | | (s, 1H), 4.21 (s, 3H), |
| | | | | | | | 3.33 (s, 3H), 2.16 |
| | | | | | | | (s, 3H), 1.37 (s, 9H). |
| G1 | Me | Cl | OH | 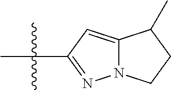 [SMILES: *c1cc2n(n1)CCC2C] | | | mixture of diastereoisomers (~1:1 ratio): 6.43 (s, 1H); 5.86 (br s, 1H); 4.95 (pseudo d, 1H); 4.18-4.09 (m, 1H); 4.05-3.95 (m, 1H); 3.35-3.23 (m, 1H); 2.80-2.68 (m, 1H); 2.18-2.08 (m, 1H); 1.96 (s, 3H); 1.33 (d, 1.5 H); 1.32 (d, 1.5 H) |
| G2 | Me | Cl | OH | 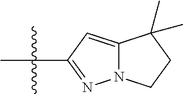 [SMILES: *c1cc2n(n1)CCC2(C)C] | | | 6.39 (s, 1H); 5.85 (s, 1H); 4.91 (d, 1H); 4.13-4.06 (m, 2H); 2.36 (dd, 2H); 1.95 (s, 3H); 1.35 (s, 3H); 1.34 (s, 3H) |
| G3 | Me | Cl | OH | 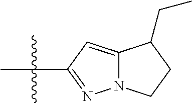 [SMILES: *c1cc2n(n1)CCC2CC] | | | mixture of diastereoisomers (~1:1 ratio): 6.44 (s, 1H); 5.88-5.85 (m, 1H); 4.88 (dd, 1H); 4.15-4.08 (m, 1H); 4.03-3.96 (m, 1H); 3.18-3.08 (m, 1H); 2.77-2.67 (m, 1H); 2.23-2.14 (m, 1H); 1.96 (s, 3H); 1.76- 1.58 (m, 2H); 1.13 (dt, 3H) |
| G4 | Me | Cl | OH | 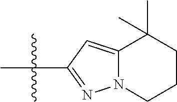 [SMILES: *c1cc2n(n1)CCCC2(C)C] | | | 6.48 (s, 1H); 5.86 (br s, 1H); 5.23 (br s, 1H); 3.95 (pseudo t, 1H); 2.10-2.03 (m, 2H); 1.94 (s, 3H); 1.72- 1.67 (m, 2H); 1.33 (s, 3H); 1.32 (s, 3H) |
| G5 | Me | Cl | OH | 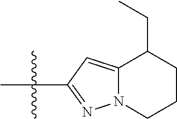 [SMILES: *c1cc2n(n1)CCCC2CC] | | | mixture of diastereomers (~1:1 ratio): 6.48 (s, 1H); 5.96 (br d, 1H); 5.07 (d, 0.5H (A)); 5.03, (d, 0.5H (B)); 4.10-4.00 (m, 1H); 3.95-3.86 (m, 1H); 2.80-2.69 (m, 1H); 2.19-2.09 (m, 1H); 2.08-1.99 (m, 1H); 1.97 (s, 3H); 1.95-1.83 (m, 1H); 1.60-1.40 (m, 3H); 0.99 (t, 1.5H (B)), 0.98 (t, 1.5H (A)) |
| G6 | Me | Cl | OH | 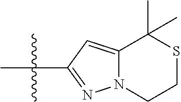 [SMILES: *c1cc2n(n1)CCSC2(C)C] | | | 6.57 (s, 1H); 5.87 (br s, 1H), 4.83 (d, 1H); 4.29-4.19 (m, 2H); 3.20-3.08 (m, 2H); 1.93 (s, 3H); 1.70 (s, 3H); 1.69 (s, 3H) |
| H1 | Me | Br | OH | Me | tBu | H | 6.48 (s, 1H); 5.87 |
| | | | | | | | (br d, 1H); 5.04 (d, |
| | | | | | | | 1H); 3.87 (s, |
| | | | | | | | 3H); 1.94 (d, 3H); |
| | | | | | | | 1.38 (s, 9H). |
| H2 | Me | Br | OH | H | tBu | H | 6.31 (s, 1H); 5.83 |
| | | | | | | | (s, 1H); 1.90 (d, |
| | | | | | | | 3H); 1.33 (s, 9H) - |
| | | | | | | | in CD3OD, no OH |
| | | | | | | | or NH detected |
| H3 | Me | Br | OH | Me | CH2CH═C(CH3)2 | H | 6.45 (s, 1H), 5.88 |
| | | | | | | | (m, 1H), 5.24 (m, |
| | | | | | | | 1H), 4.93 (d, 1H), |
| | | | | | | | 3.69 (s, 3H), 3.29 |
| | | | | | | | (m, 2H), 1.95 (s, |
| | | | | | | | 3H), 1.76 (s, 3H), |
| | | | | | | | 1.71 (s, 3H). |
| H4 | Me | Br | OH | Me | CF3 | H | 7.08 (s, 1H); 5.93 |
| | | | | | | | (d, 1H); 4.39 (d, |
| | | | | | | | 1H); 3.92 (s, 3H); |
| | | | | | | | 1.93 (s, 3H) |
| H5 | Me | Br | OH | Me | tBu | CN | 5.83 (d, 1H), 4.0 (s, |
| | | | | | | | 3H), 3.65 (d, 1H), |
| | | | | | | | 1.96 (s, 3H), 1.55 |
| | | | | | | | (s, 9H). |
| H6 | Et | Br | OH | Me | tBu | CN | 5.84 (d, 1H), 3.39 |
| | | | | | | | (d, 1H), 2.24 (q, |
| | | | | | | | 2H), 1.59 (s, 9H), |
| | | | | | | | 1.18 (t, 3H). |
| H7 | Me | Br | OH | 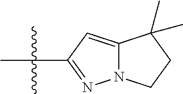 [SMILES: *c1cc2n(n1)CCC2(C)C] | | | 6.39 (s, 1H); 5.90 (s, 1H); 5.16 (br s, 1H); 4.08 (t, 2H); 2.44 (t, 2H); 1.95 (s, 3H); 1.37 (s, 3H); 1.36 (s, 3H) |
| H8 | Me | Br | OH | 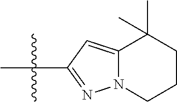 [SMILES: *c1cc2n(n1)CCCC2(C)C] | | | 6.48 (s, 1H); 5.89 (s, 1H); 5.12 (br s, 1H); 3.95 (br d, 2H); 2.10-2.02 (m, 2H); 1.94 (s, 3H); 1.72-1.68 (m, 2H); 1.33 (s, 3H); 1.32 (s, 3H). |
| H9 | Me | Br | OH | 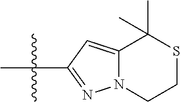 [SMILES: *c1cc2n(n1)CCSC2(C)C] | | | 6.56 (s, 1H); 5.90 (br s, 1H); 4.92 (br d, 1H); 4.22 (br t, 2H); 3.17-3.10 (m, 2H); 1.94 (s, 3H); 1.69 (s, 3H); 1.68 (s, 3H). |
| H10 | Me | Br | OH | 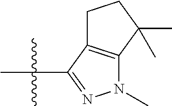 [SMILES: *c1nn(C)c2c1CCC2(C)C] | | | 6.56 (br s, 1H); 5.16 (br d, 1H); 3.58 (s, 3H); 2.92- 2.78 (m, 2H); 2.31- 2.26 (m, 2H); 1.93 (s, 3H); 1.32 (s, 3H); 1.31 (s, 3H). |
| I1 | Me | OMe | OH | H | tBu | H | 6.30 (br s, 1H); |
| | | | | | | | 5.93 (br s, 1H); |
| | | | | | | | 4.10 (s, 3H); 1.79 |
| | | | | | | | (s, 3H); 1.32 (s, |
| | | | | | | | 9H), no OH or NH |
| | | | | | | | detected, in CD₃OD |
| I2 | Me | OMe | OH | Me | tBu | H | 6.49 (br s, 1H); |
| | | | | | | | 5.92 (br s, 1H); |
| | | | | | | | 5.37 (br s, 1H); |
| | | | | | | | 4.10 (s, 3H); 3.78 |
| | | | | | | | (s, 3H); 1.85 (s, |
| | | | | | | | 3H); 1.37 (s, 9H). |
| I3 | Me | OMe | OH | Me | CF₃ | H | 6.36 (s, 1H); 5.49 |
| | | | | | | | (d, 1H); 4.64 (d, |
| | | | | | | | 1H); 4.09 (s, 3H); |
| | | | | | | | 3.72 (s, 3H); 1.87 |
| | | | | | | | (s, 3H). |
| I4 | Me | OMe | OH | 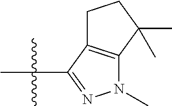 [SMILES: *c1nn(C)c2c1CCC2(C)C] | | | 6.39 (s, 1H); 5.92 (s, 1H), 5.11 (br s, 1H); 4.08 (s, 3H); 4.04 (t, 2H); 2.35 (t, 2H); 1.88 (s, 3H); 1.37 (s, 3H); 1.36 (s, 3H). |
| I5 | Me | OMe | OH | 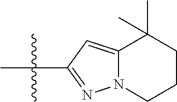 [SMILES: *c1cc2n(n1)CCCC2(C)C] | | | 6.50 (s, 1H); 5.92 (s, 1H); 5.15 (br s, 1H); 4.09 (s, 3H); 3.96 (t, 2H); 2.08- 2.01 (m, 2H); 1.86 (s, 3H); 1.69-1.65 (m, 2H); 1.33 (s, 3H); 1.32 (s, 3H). |
| J1 | OMe | Cl | OH | Me | tBu | H | 6.46 (s, 1H), 5.81 (m, 1H), 4.97 (br m, 1H), 4.21 (s, 3H), 3.87 (s, 3H), 1.38 (s, 9H). |

### Example 9

### Herbicidal Action

### Post-Emergence Herbicidal Activity

Seeds of a variety of test species were sown in standard soil in pots. After 8 days cultivation (post-emergence) under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethelyene sorbitan monolaurate, CAS RN 9005-64-5). The test plants were then grown in a glasshouse under controlled conditions (at 24/16° C., day/night; 14 hours light; 65% humidity) and watered twice daily. After 13 days, the test was evaluated (5=total damage to plant; 0=no damage to plant). Results are shown in Table 2.

TABLE 2
Application post-emergence

| Compound | Rate | | | | | | |
|---|---|---|---|---|---|---|---|
| Number | (g/ha) | ABUTH | AMARE | SETFA | ALOMY | ECHCG | ZEAMX |
| A1 | 250 | 5 | 2 | 4 | 4 | 5 | 0 |
| A2 | 1000 | 4 | 1 | 2 | 1 | 2 | 0 |
| A3 | 250 | 5 | 5 | 4 | 5 | 5 | 1 |
| A4 | 1000 | 5 | 4 | 4 | 4 | 5 | 1 |
| A5 | 1000 | 5 | 3 | 4 | 4 | 2 | 2 |
| A6 | 1000 | 2 | 3 | 2 | 1 | 2 | 1 |
| A7 | 1000 | 5 | 5 | 5 | 3 | 5 | 1 |
| B1 | 1000 | 5 | 5 | 1 | 2 | 2 | 1 |
| B2 | 1000 | 5 | 5 | 5 | 5 | 5 | 2 |
| B3 | 1000 | 5 | 5 | 4 | 5 | 5 | 1 |
| B4 | 1000 | 5 | 5 | 3 | 4 | 4 | 0 |
| B5 | 250 | 5 | 5 | 4 | 4 | 5 | 3 |
| B6 | 1000 | 5 | 5 | 5 | 5 | 5 | 2 |
| B7 | 1000 | 4 | 3 | 5 | 0 | 1 | 2 |
| B8 | 1000 | 5 | 5 | 5 | 3 | 4 | 1 |
| B9 | 1000 | 5 | 5 | 5 | 4 | 5 | 1 |
| B10 | 1000 | 2 | 0 | 3 | 0 | 2 | 0 |
| B11 | 1000 | 0 | 0 | 0 | 0 | 0 | 0 |
| B12 | 1000 | 1 | 0 | 0 | 0 | 0 | 0 |
| B13 | 1000 | 5 | 3 | 4 | 3 | 2 | 0 |
| B14 | 1000 | 5 | 4 | 4 | 3 | 4 | 1 |
| B15 | 1000 | 5 | 5 | 5 | 4 | 5 | 2 |
| B16 | 1000 | 0 | 0 | 0 | 0 | 0 | 0 |
| B17 | 1000 | 4 | 3 | 5 | 2 | 4 | 1 |
| B18 | 1000 | 5 | 5 | 5 | 4 | 5 | 4 |
| B19 | 1000 | 4 | 4 | 4 | 2 | 3 | 0 |
| B20 | 1000 | 5 | 5 | 5 | 3 | 4 | 0 |
| B21 | 1000 | 4 | 5 | 5 | 5 | 5 | 1 |
| B22 | 1000 | 1 | 0 | 3 | 1 | 2 | 1 |
| B23 | 1000 | 4 | 5 | 4 | 3 | 5 | 1 |
| B24 | 1000 | 5 | 5 | 5 | 3 | 4 | 1 |
| B25 | 1000 | 5 | 5 | 5 | 4 | 5 | 2 |
| B26 | 1000 | 5 | 5 | 5 | 4 | 5 | 4 |
| B27 | 1000 | 4 | 1 | 3 | 1 | 0 | 0 |
| B28 | 1000 | 4 | 4 | 3 | 2 | 3 | 1 |
| B29 | 1000 | 5 | 5 | 5 | 4 | 5 | 3 |
| B30 | 1000 | 5 | 3 | 4 | 4 | 5 | 1 |
| B31 | 1000 | 0 | 3 | 1 | 1 | 1 | 1 |
| B32 | 1000 | 5 | 3 | 4 | 2 | 3 | 0 |
| C1 | 1000 | 3 | 5 | 0 | 3 | 4 | 1 |
| C2 | 1000 | 5 | 4 | 4 | 3 | 4 | 0 |
| C3 | 1000 | 5 | 5 | 5 | 4 | 5 | 1 |
| C4 | 1000 | 0 | 1 | 1 | 0 | 1 | 1 |
| C5 | 1000 | 2 | 1 | 0 | 0 | 0 | 3 |
| C6 | 1000 | 1 | 2 | 2 | 0 | 0 | 1 |
| C7 | 1000 | 3 | 0 | 1 | 0 | 0 | 0 |
| C8 | 1000 | 0 | 1 | 0 | 0 | 0 | 0 |
| C9 | 1000 | 1 | 1 | 0 | 0 | 1 | 1 |
| D1 | 1000 | 3 | 4 | 1 | 2 | 3 | 0 |
| D2 | 1000 | 3 | 0 | 2 | 1 | 2 | 0 |
| D3 | 1000 | 1 | 0 | 1 | 0 | 2 | 1 |
| D4 | 1000 | 4 | 2 | 4 | 3 | 2 | 0 |
| E1 | 1000 | 4 | 1 | 5 | 4 | 5 | 0 |
| E2 | 1000 | 4 | 5 | 4 | 4 | 5 | 1 |
| F1 | 1000 | 5 | 5 | 5 | 4 | 5 | 3 |
| F2 | 1000 | 5 | 5 | 5 | 4 | 5 | 1 |
| F3 | 1000 | 5 | 5 | 5 | 5 | 5 | 3 |
| G1 | 1000 | 5 | 5 | 4 | 4 | 5 | 2 |
| G2 | 250 | 5 | 5 | 5 | 5 | 5 | 4 |
| G3 | 1000 | 5 | 5 | 5 | 4 | 4 | 2 |
| G4 | 1000 | 5 | 5 | 5 | 5 | 5 | 2 |
| G5 | 1000 | 5 | 5 | 4 | 4 | 4 | 0 |
| G6 | 1000 | 5 | 5 | 4 | 4 | 4 | 0 |
| H1 | 1000 | 5 | 5 | 3 | 4 | 5 | 1 |
| H2 | 1000 | 5 | 5 | 4 | 3 | 2 | 2 |
| H3 | 1000 | 0 | 0 | 0 | 0 | 0 | 0 |
| H4 | 1000 | 5 | 5 | 1 | 5 | 2 | 0 |
| H5 | 1000 | 2 | 2 | 1 | 1 | 1 | 0 |
| H6 | 1000 | 1 | 2 | 2 | 2 | 1 | 0 |
| H7 | 1000 | 5 | 4 | 4 | 5 | 3 | 1 |
| H8 | 1000 | 5 | 5 | 4 | 5 | 4 | 0 |
| H9 | 1000 | 4 | 4 | 3 | 5 | 3 | 0 |
| H10 | 1000 | 5 | 5 | 5 | 5 | 4 | 3 |
| I1 | 250 | 5 | 5 | 5 | 3 | 3 | 1 |
| I2 | 250 | 5 | 5 | 4 | 4 | 3 | 1 |
| I3 | 1000 | 5 | 5 | 5 | 5 | 5 | 4 |
| I4 | 1000 | 5 | 5 | 5 | 5 | 5 | 3 |
| I5 | 1000 | 5 | 5 | 5 | 5 | 5 | 4 |
| J1 | 1000 | 5 | 5 | 5 | 5 | 5 | 3 |

ABUTH = Abutilon theophrasti; AMARE = Amaranthus retroflexus; SETFA = Setaria faberi; ALOMY = Alopecurus myosuroides; ECHCG = Echinochloa crus-galli; ZEAMX = Zea mays.

1. A herbicidal compound of formula (I) 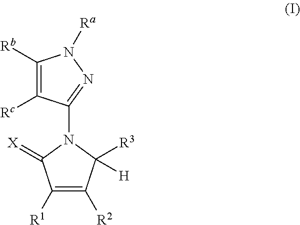
wherein
X is selected from S and O;
R^{a} is selected from hydrogen, C₁-C₆ alkyl and C₁-C₆ haloalkyl;
R^{b} is selected from hydrogen, formyl, hydroxyl, halogen, nitro, cyano, C₁-C₆ alkyl, C₁-C₆ cyanoalkyl, C₃-C₆ cycloalkyl, C₃-C₆ cyanocycloalkyl, C₁-C₆ haloalkyl, C₁-C₆ alkylthio, C₁-C₆ alkoxy, C₁-C₆ alkoxy C₁-C₆ alkyl, C₁-C₆ alkthio C₁-C₆ alkyl, C₁-C₆ cyanoalkoxy, C₁-C₆ haloalkoxy, C₁-C₆ alkoxy C₁-C₆ alkoxy, C₂-C₆ alkenyl, C₂-C₆ alkynyl, C₂-C₆ cyanoalkenyl, C₂-C₆ cyanoalkynyl, C₂-C₆ alkenyloxy, C₂-C₆ alkynyloxy, C₂-C₆ haloalkenyl, C₂-C₆ haloalkynyl, C₂-C₆ haloalkenyloxy, C₂-C₆ haloalkynyloxy, C₁-C₆ alkoxy C₂-C₆ alkenyl, C₁-C₆ alkoxy C₂-C₆ alkynyl, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylsulfonyloxy, C₁-C₆ alkylcarbonyl, C₁-C₆ haloalkylcarbonyl, C₂-C₆ alkenylcarbonyl, C₂-C₆ alkynylcarbonyl, C₂-C₆ haloalkenylcarbonyl, C₂-C₆ haloalkynylcarbonyl, tri C₁-C₆ alkylsilyl C₂-C₆ alkynyl, C₁-C₆ alkylamido, a group R⁵R⁶N—, a group R⁵C(O)N(R⁶)—, a group R⁵S(O₂)N(R⁶)—, a group R⁵R⁶NSO₂—, a C₆-C₁₀ aryl group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, a C₆-C₁₀ aryloxy group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, a C₆-C₁₀ benzyl group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, a C₆-C₁₀ benzyloxy group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, a C₃-C₆ heterocyclyl group optionally substituted by from 1 to 3 groups independently selected from C₁-C₄ alkyl and a C₃-C₆ cycloalkyl group optionally substituted with from 1 to 3 groups independently selected from halogen or C₁-C₆ alkyl;
and wherein when R^{b} is C₂-C₆ alkynyl, C₂-C₆ cyanoalkynyl, C₂-C₆ haloalkynyl or C₁-C₆ alkoxy C₂-C₆ alkynyl, the alkynyl group is not directly attached to the pyrazole ring;
R^{c} is selected from hydrogen, halogen, cyano, C₁-C₆ alkyl or C₁-C₆ haloalkyl;
or R^{a} and R^{b} together with the nitrogen and carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl;
or R^{b} and R^{c} together with the carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl;
R¹ is C₁-C₆ alkyl, C₁-C₆ haloalkyl or C₁-C₃ alkoxy and R² is halogen or C₁-C₃ alkoxy with the proviso that R¹ and R² are not both C₁-C₃ alkoxy;
R³ is selected from halogen, hydroxyl, or any one of the following groups 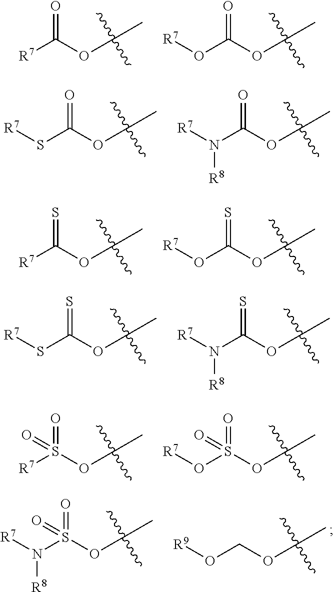
R⁵ and R⁶ are independently selected from hydrogen, C₁-C₆ alkyl, C₁-C₆ haloalkyl, C₂-C₆ alkenyl, C₂-C₆ alkynyl, or R⁵ and R⁶ together with the carbon atoms to which they are attached form a 3-6 membered saturated or partially unsaturated ring optionally comprising from 1 to 3 heteroatoms independently selected from S, O and N and optionally substituted with from 1 to 3 groups independently selected from halogen or C₁-C₆ alkyl;
R⁷ and R⁸ are independently selected from hydrogen, C₁-C₆ alkyl, C₁-C₆ haloalkyl, C₂-C₆ alkenyl, C₂-C₆ alkynyl, a C₅-C₁₀ heteroaryl group which can be mono- or bicyclic comprising from 1 to 4 heteroatoms independently selected from N, O and S and optionally substituted with 1 to 3 groups independently selected from halogen, C₁-C₃ alkyl, C₁-C₃ haloalkyl and C₁-C₃ alkoxy, a C₆-C₁₀ aryl group optionally substituted with 1 to 3 groups independently selected from halogen, nitro, cyano, C₁-C₃ alkyl, C₁-C₃ alkoxy, C₁-C₃ haloalkyl and C₁-C₃ haloalkoxy, or R⁷ and R⁸ together with the atoms to which they are attached form a 3-6 membered saturated or partially unsaturated ring optionally comprising from 1 to 3 heteroatoms independently selected from S, O and N and optionally substituted with from 1 to 3 groups independently selected from halogen or C₁-C₆ alkyl;
R⁹ is selected from C₁-C₆ alkyl or benzyl optionally substituted with 1 to 3 groups independently selected from halogen, nitro, cyano, C₁-C₃ alkyl, C₁-C₃ alkoxy, C₁-C₃ haloalkyl, and C₁-C₃ haloalkoxy;
or an N-oxide or salt form thereof.

2. The compound of claim 1, wherein X is O.

3. The compound of claim 1, wherein R^{a} is selected from hydrogen, methyl, ethyl, C₁-C₂ haloalkyl or R^{a} and R^{b} together with the nitrogen and carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl.

4. (canceled)

5. The compound of claim 1, wherein R^{b} is selected from hydrogen, halogen, C₁-C₆ alkyl, C₃-C₆ cycloalkyl, C₁-C₆ haloalkyl, C₂-C₆ alkenyl, C₁-C₆ cyanoalkyl, C₁-C₆ alkylthio, C₁-C₆ alkylsulfinyl, C₁-C₆ alkylsulphonyl, C₁-C₅ alkoxy C₁-C₆ alkyl, a C₆-C₁₀ aryl group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, a C₃-C₆ heteroaryl group optionally substituted by from 1 to 3 groups independently selected from C₁-C₄ alkyl, a C₆-C₁₀ benzyl group optionally substituted by from 1 to 3 groups independently selected from halogen, nitro, cyano, C₁-C₃ alkyl, C₁-C₃ alkoxy, C₁-C₃ haloalkyl and C₁-C₃ haloalkoxy, or R^{a} and R^{b} together with the nitrogen and carbon atoms to which they are attached form a 3-7 membered saturated or partially unsaturated ring optionally comprising 1 to 3 heteroatoms independently selected from S, O and N and optionally substituted with 1 to 3 groups independently selected from C₁-C₆ alkyl or R^{b} and R^{c} together with the carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl.

6. (canceled)

7. (canceled)

8. (canceled)

9. (canceled)

10. The compound of claim 1, wherein R^{c} is selected from hydrogen, methyl, chloro or cyano or R^{b} and R^{c} together with the carbon atoms to which they are attached form a 3-7 membered saturated or partially unsaturated ring optionally comprising from 1 to 3 heteroatoms independently selected from S, O and N and optionally substituted with from 1 to 3 groups independently selected from halogen, C₁-C₆ alkyl and C₁-C₆ haloalkyl.

11. (canceled)

12. The compound of claim 1, wherein R¹ is selected from methyl, ethyl, methoxy or ethoxy, with the proviso that when R¹ is methoxy or ethoxy, R² is not methoxy or ethoxy.

13. The compound of claim 1, wherein R² is selected from bromo, chloro, methoxy or ethoxy, with the proviso that when R¹ is methoxy or ethoxy, R² is not methoxy or ethoxy.

14. (canceled)

15. The compound of claim 1, wherein R³ is selected from halogen, hydroxyl, C₁-C₆ alkoxycarbonyloxy or aryloxycarbonyloxy wherein the aryl group may be substituted with 1 to 3 groups independently selected from halogen, nitro, cyano, C₁-C₃ alkyl, C₁-C₃ alkoxy, C₁-C₃ haloalkyl and C₁-C₃ haloalkoxy.

16. (canceled)

17. (canceled)

18. A herbicidal composition comprising a compound of formula I as defined in claim 1 together with at least one agriculturally acceptable adjuvant or diluent.

19. A composition according to claim 18 which comprises a further herbicide in addition to the compound of formula I.

20. A composition according to claim 18 which comprises a safener.

21. (canceled)

22. A method of controlling weeds in crops of useful plants, comprising applying to said weeds or to the locus of said weeds, or to said useful plants or to the locus of said useful plants, a compound of formula I as defined in claim 1.

23. The method of claim 22, wherein said useful plants are maize plants.
